(19) United States
(12) Patent Application Publication
(10) Pub. No.: US 2010/0189683 A1
(43) Pub. Date: Jul. 29, 2010

(54) Pulsatile Dosing of Gossypol for Treatment of Disease

(75) Inventors: Jon T. Holmlund, Carlsbad, CA (US); Mel Sorensen, Malvern, PA (US); Lance Leopold, Dresher, PA (US); Dajun Yang, Rockville, MD (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Ascenta Therapeutics, Inc., Malvern, PA (US)

(21) Appl. No.: 12/635,481

(22) Filed: Dec. 10, 2009

Related U.S. Application Data
(63) Continuation-in-part of application No. 12/332,039, filed on Dec. 10, 2008.
Application No. 12/332,039 is a continuation of application No. PCT/US2009/064295, filed on Nov. 13, 2009.
Application No. 12/332,039 is a continuation-in-part of application No. 12/292,282, filed on Nov. 14, 2008, now abandoned.

(51) Int. Cl. A61K 38/20; A61K 31/11; A61K 38/00; A61K 39/395; A61K 31/203; A61K 31/519; A61K 31/53; A61K 31/661; A61K 31/45; A61K 31/4196; A61K 31/285; A61K 38/50; A61K 31/7036; A61K 31/52; A61K 39/07; A61K 31/192; A61K 31/167; A61K 38/14; A61K 31/69; A61K 31/255; A61K 31/4152; A61K 31/505; A61K 31/282; A61K 33/24; A61K 31/7068; A61K 38/16; A61K 31/573; A61K 38/21; A61K 33/12; A61K 31/337; A61K 31/437; A61K 31/517; A61K 31/454; A61P 35/00

(52) U.S. Cl. 424/85.2; 514/700; 514/2; 424/133.1; 514/557; 514/262.1; 514/245; 514/114; 514/328; 514/383; 424/623; 424/94.6; 514/43; 514/263.2; 424/184.1; 514/569; 514/618; 514/8; 514/64; 514/517; 514/404; 514/269; 514/492; 424/649; 514/49; 514/12; 514/179; 424/85.7; 424/683; 514/449; 514/283; 514/266.4; 514/177; 514/323; 514/285

(57) ABSTRACT

This invention relates to pulsatile dose administration of gossypol or pharmaceutical compositions thereof for treating diseases, disorders and conditions responsive to gossypol, inhibiting the activity of anti-apoptotic Bcl-2 family proteins, inducing apoptosis in cells and increasing the sensitivity of cells to inducers of apoptosis.

### BACKGROUND OF THE INVENTION

1. Field of the Invention

This invention is in the field of medicinal chemistry. In particular, the invention relates to pulsatile dose administration of gossypol or pharmaceutical compositions thereof for treating diseases, disorders and conditions responsive to gossypol, inhibiting the activity of anti-apoptotic Bcl-2 family proteins, inducing apoptosis in cells and increasing the sensitivity of cells to inducers of apoptosis.

2. Related Art

The aggressive cancer cell phenotype is the result of a variety of genetic and epigenetic alterations leading to deregulation of intracellular signaling pathways (Ponder, Nature 411:336 (2001)). The commonality for all cancer cells, however, is their failure to execute an apoptotic program, and lack of appropriate apoptosis due to defects in the normal apoptosis machinery is a hallmark of cancer (Lowe et al., Carcinogenesis 21:485 (2000)). Most of the current cancer therapies, including chemotherapeutic agents, radiation (radiotherapeutic agents), and immunotherapy, work by indirectly inducing apoptosis in cancer cells. The inability of cancer cells to execute an apoptotic program due to defects in the normal apoptotic machinery is thus often associated with an increase in resistance to chemotherapy, radiation, or immunotherapy-induced apoptosis. Primary or acquired resistance of human cancer of different origins to current treatment protocols due to apoptosis defects is a major problem in current cancer therapy (Lowe et al., Carcinogenesis 21:485 (2000); Nicholson, Nature 407:810 (2000)). Accordingly, current and future efforts towards designing and developing new molecular target-specific anticancer therapies to improve survival and quality of life of cancer patients must include strategies that specifically target cancer cell resistance to apoptosis. In this regard, targeting crucial negative regulators that play a central role in directly inhibiting apoptosis in cancer cells represents a highly promising therapeutic strategy for new anticancer drug design.

Two classes of central negative regulators of apoptosis have been identified. The first class of regulators is the inhibitor of apoptosis proteins (IAPs) (Deveraux et al., Genes Dev. 13:239 (1999); Salvesen et al., Nat. Rev. Mol. Cell. Biol. 3:401 (2002)). IAP proteins potently suppress apoptosis induced by a large variety of apoptotic stimuli, including chemotherapeutic agents, radiation, and immunotherapy in cancer cells.

The second class of central negative regulators of apoptosis is the Bcl-2 family of proteins (Adams et al., Science 281:1322 (1998); Reed, Adv. Pharmacol. 41:501 (1997); Reed et al., J. Cell. Biochem. 60:23 (1996)). Bcl-2 is the founding member of the family and was first isolated as the product of an oncogene. The Bcl-2 family now includes both anti-apoptotic molecules such as Bcl-2, Bcl-X_{L}, and Mcl-1 and pro-apoptotic molecules such as Bax, Bak, Bid, and Bad. Bcl-2 and Bcl-X_{L} are overexpressed in many types of human cancer (e.g., breast, prostate, colorectal, lung, etc.), including Non-Hodgkin's lymphoma, which is caused by a chromosomal translocation (t14, 18) that leads to overexpression of Bcl-2. This suggests that many cancer cell types depend on the elevated levels of Bcl-2 and/or Bcl-X_{L} to survive the other cellular derangements that simultaneously both define them as cancerous or pre-cancerous cells and cause them to attempt to execute the apoptosis pathway. Also, increased expression of Bcl-2 family proteins has been recognized as a basis for the development of resistance to cancer therapeutic drugs and radiation that act in various ways to induce cell death in tumor cells.

Bcl-2 and Bcl-X_{L} are thought to play a role in tumor cell migration and invasion, and therefore, metastasis (Amberger et al., Cancer Res. 58:149 (1998); Wick et al., FEBS Lett. 440:419 (1998); Mohanam et al., Cancer Res. 53:4143 (1993); Pedersen et al., Cancer Res. 53:5158 (1993)). Bcl-2 family proteins appear to provide tumor cells with a mechanism for surviving in new and non-permissive environments (e.g., metastatic sites), and contribute to the organospecific pattern of clinical metastatic cancer spread (Rubio, Lab Invest. 81:725 (2001); Fernandez et al., Cell Death Differ. 7:350 (2000)). Anti-apoptotic proteins such as Bcl-2 and/or Bcl-X_{L} are also thought to regulate cell-cell interactions, for example through regulation of cell surface integrins (Reed, Nature 387:773 (1997); Frisch et al., Curr. Opin. Cell Biol. 9:701 (1997); Del Bufalo et al., FASEB J. 11:947 (1997)).

Therapeutic strategies for targeting Bcl-2 and Bcl-X_{L} in cancer to restore cancer cell sensitivity and overcome resistance of cancer cells to apoptosis have been extensively reviewed (Adams et al., Science 281:1322 (1998); Reed, Adv. Pharmacol. 41:501 (1997); Reed et al., J. Cell. Biochem. 60:23 (1996)).

Gossypol is a naturally occurring double biphenolic compound derived from crude cotton seed oil (Gossypium sp.). Human trials of gossypol as a male contraceptive have demonstrated the safety of long term administration of these compounds (Wu, Drugs 38:333 (1989)). Gossypol has more recently been shown to have some anti-proliferative effects (Flack et al., J. Clin. Endocrinol. Metab. 76:1019 (1993); Bushunow et al., J. Neuro-Oncol. 43:79, (1999); Van Poznak et al., Breast Cancer Res. Treat. 66:239 (2001)). Gossypol and its derivatives recently have been shown to be potent inhibitors of Bcl-2, Bcl-X_{L}, and Mcl-1, and to have strong anti-cancer activity (U.S. Patent Application No. 2003/0008924).

### SUMMARY OF THE INVENTION

The present invention relates to pulsatile dose administration of gossypol, i.e., (±)-gossypol (I), (−)-gossypol (II) or (+)-gossypol. It has surprisingly been found that pulsatile dose administration of gossypol provides clinical efficacy coupled with a reduction in adverse events. Gossypol and pharmaceutical compositions thereof are useful for treating hyperproliferative and other diseases, inhibiting the activity of anti-apoptotic Bcl-2 family proteins, inducing apoptosis in cells and increasing the sensitivity of cells to inducers of apoptosis.

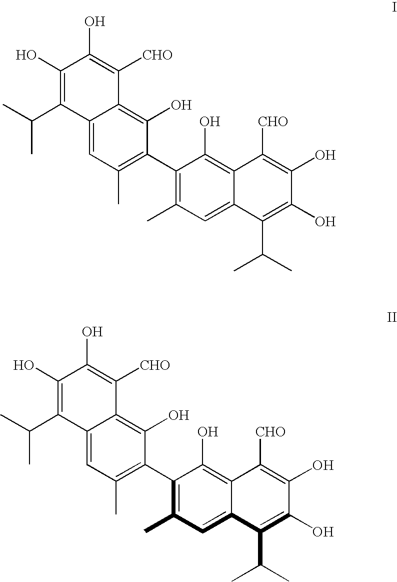

The present invention contemplates that pulsatile dose administration of gossypol to patients suffering from cancer and other diseases will expose patients to therapeutically effective amounts of gossypol and will minimize unwanted adverse events. Gossypol inhibits the function(s) of anti-apoptotic Bcl-2 family proteins and will kill cancer cells or supporting cells outright (those cells whose continued survival is dependent on the overactivity of Bcl-2 family proteins) and/or render such cells as a population more susceptible to the cell death-inducing activity of chemotherapeutic and/or radiotherapeutic agents. The present invention contemplates that gossypol administered by pulsatile dosing will satisfy an unmet need for the treatment of multiple cancer types, either when administered as monotherapy to induce apoptosis in cancer cells dependent on anti-apoptotic Bcl-2 family proteins function, or when administered in a temporal relationship with other cell death-inducing chemotherapeutic and/or radiotherapeutic agents so as to render a greater proportion of the cancer cells or supportive cells susceptible to executing the apoptosis program compared to the corresponding proportion of cells in a patient treated only with the chemotherapeutic and/or radiotherapeutic agent alone. The present invention further contemplates that gossypol administered by pulsatile dosing will induce apoptosis and/or render cells more sensitive to induction of apoptosis in other diseases or conditions characterized by dysregulation of apoptosis.

In certain embodiments of the invention, it is expected that combination treatment of patients with a therapeutically effective amount of gossypol administered by pulsatile dosing and one or more additional therapeutic agents will produce a greater tumor response and clinical benefit in such patients compared to those treated with either gossypol or one or more therapeutic agents alone. Put another way, because gossypol administered by pulsatile dosing lowers the apoptotic threshold of all cells that express anti-apoptotic Bcl-2 family proteins, the proportion of cells that successfully execute the apoptosis program in response to the apoptosis inducing activity of therapeutic agents, such as anticancer drugs, will be increased. Alternatively, gossypol administered by pulsatile dosing is expected to allow administration of a lower, and therefore less toxic and more tolerable, dose of an anticancer agent to produce the same tumor response/clinical benefit as the conventional dose of the anticancer agent alone. Since the doses for all approved anticancer agents are known, the present invention contemplates combination therapies with various combinations of known anticancer agents with gossypol administered by pulsatile dosing. Also, since gossypol administered by pulsatile dosing acts at least in part by inhibiting anti-apoptotic Bcl-2 family proteins, the exposure of cancer cells and supporting cells to therapeutically effective amounts of gossypol can be temporally linked to coincide with the attempts of cells to execute the apoptosis program in response to the anticancer agent. Thus, in some embodiments, administering gossypol by pulsatile dosing in connection with certain temporal relationships, will provide especially efficacious therapeutic practices.

Gossypol administered by pulsatile dosing is useful for the treatment, amelioration, or prevention of disorders responsive to induction of apoptotic cell death, e.g., disorders characterized by dysregulation of apoptosis, including hyperproliferative diseases such as cancer. In certain embodiments, gossypol administered by pulsatile dosing can be used to treat, ameliorate, or prevent cancer that is characterized by resistance to cancer therapies (e.g., those which are chemoresistant, radiation resistant, hormone resistant, and the like). In one embodiment, the cancer is chemoresistant to treatment with a taxane, i.e., docetaxel or paclitaxel. In another embodiment, the cancer is chemoresistant to treatment with an anticancer agent selected from the group consisting of topotecan HCl, erlotinib, lenalidomide, cisplatin, erbitux, and oxaplatin. In one embodiment, the chemoresistant cancer is prostate cancer. In another embodiment, the cancer is non-small cell lung cancer. In another embodiment, the chemoresistant cancer is selected from the group consisting of breast cancer, glioma, myeloma, chronic lymphocytic leukemia, laryngeal cancer, head and neck cancer, ovarian cancer, and colorectal cancer. In additional embodiments, gossypol administered by pulsatile dosing can be used to treat, ameliorate, or prevent metastatic cancer or squamous cell cancer. In other embodiments, gossypol administered by pulsatile dosing can be used to treat hyperproliferative and other diseases characterized by overexpression of anti-apoptotic Bcl-2 family proteins. In other embodiments, gossypol administered by pulsatile doing can be used to modulate spermicidal activity (e.g., function as a male contraceptive or antifertility agent), treat malaria, microbial or viral disease (e.g., inhibit the growth of the HIV virus as a treatment for AIDS), treat obesity, skin disorders or baldness, inhibit growth of endothelial cells, inhibit vascularization or neovascularization, treat arthritic conditions, neovascular-based dermatological conditions, diabetic retinopathy, Kaposi's sarcoma, age-related macular degeneration, restenosis, telangiectasia, glaucoma, keloids, corneal graft rejection, wound granularization, angiofibroma, Osler-Webber syndrome, myocardial angiogenesis, psoriatic arthritis or scleroderma, inhibit DNA synthesis or DNA polymerase activity and treat gynecological disorders (e.g., endometriosis) or diabetic complications.

In one particular embodiment, the invention relates to a method of treating or ameliorating cancer comprising administering to a patient in need thereof (−)-gossypol by pulsatile dose administration wherein about 20 mg to about 60 mg of the (−)-gossypol is orally administered to the patient twice-a-day on day 1, day 2, and day 3 of a treatment cycle.

In another particular embodiment, the invention relates to a method of treating or ameliorating prostate cancer or non-small cell lung cancer comprising administering to a patient in need thereof (−)-gossypol by pulsatile dose administration in combination with docetaxel, wherein about 40 mg of the (−)-gossypol is orally administered to the patient twice-a-day on day 1, day 2, and day 3 of a 21-day treatment cycle and about 75 mg/m² of the docetaxel is intravenously administered to the patient on day 1 of the treatment cycle.

In another particular embodiment, the invention relates to a method of reducing the number of one or more adverse events, the severity of one or more adverse events, or combination thereof, in a patient undergoing cancer therapy comprising administering gossypol to the patient.

### BRIEF DESCRIPTION OF THE DRAWINGS/FIGURES

FIG. 1 is a chart showing pulsatile dose administration schedules of gossypol.

FIG. 2 is a line graph showing the in vivo activity of (−)-gossypol acetic acid co-crystal via pulse dose administration in combination with docetaxel.

FIG. 3 is a line graph showing the in vivo activity of (−)-gossypol acetic acid co-crystals via pulse dose administration in combination with docetaxel.

FIG. 4 is a waterfall plot showing PSA response in chemo-naive patients after treatment with (−)-gossypol acetic acid co-crystals.

FIG. 5 is a line graph showing PSA response versus time in chemo-naive patients after treatment with (−)-gossypol acetic acid co-crystals.

FIG. 6 is a series of four images showing tumor size before and after treatment with (−)-gossypol acetic acid co-crystals.

FIG. 7 is a waterfall plot showing PSA response in docetaxel (taxotere)-resistant patients after treatment with (−)-gossypol acetic acid co-crystals.

FIG. 8 is a line graph showing PSA response kinetics in a docetaxel (taxotere)-resistant patient after treatment with (−)-gossypol acetic acid co-crystals.

FIG. 9 is a line graph showing PSA response kinetics in a docetaxel (taxotere)-resistant patient after treatment with (−)-gossypol acetic acid co-crystals.

FIG. 10 is a line graph showing PSA response kinetics in a docetaxel (taxotere)-resistant patient after treatment with (−)-gossypol acetic acid co-crystals.

FIG. 11 is two tables showing the dosing schedules of: 1) AT-101 and docetaxel; and 2) placebo and docetaxel.

FIG. 12 is a line graph showing progression free survival Kaplan-Meier curves for patients treated with AT-101 and docetaxel or placebo and docetaxel.

FIG. 13 is a line graph showing progression free survival Kaplan-Meier curves for patients treated with AT-101 and docetaxel or placebo and docetaxel.

FIG. 14 is a line graph showing overall survival Kaplan-Meier curves for patients treated with AT-101 and docetaxel or placebo and docetaxel.

FIG. 15 is a line graph showing overall survival Kaplan-Meier curves for patients with squamous cell histology treated with AT-101 and docetaxel or placebo and docetaxel.

FIG. 16 is a waterfall plot showing PSA response in docetaxel (taxotere)-refractory CRPC patients after treatment with AT-101.

FIG. 17 is a waterfall plot showing PSA and RECIST correlation in docetaxel (taxotere)-refractory CRPC patients after treatment with AT-101.

FIG. 18 is a line graph showing PSA response kinetics in a docetaxel (taxotere)-resistant patient after treatment with (−)-gossypol acetic acid co-crystals (DP=docetaxel-prednisone regimen; ADP=AT-101 added to DP).

FIG. 19 is a line graph showing PSA response kinetics in a docetaxel (taxotere)-resistant patient after treatment with (−)-gossypol acetic acid co-crystals (DP=docetaxel-prednisone regimen; ADP=AT-101 added to DP).

FIG. 20 is a line graph showing PSA response kinetics in a docetaxel (taxotere)-resistant patient after treatment with (−)-gossypol acetic acid co-crystals (DP=docetaxel-prednisone regimen; ADP=AT-101 added to DP).

FIG. 21 is a line graph showing PSA response kinetics in a docetaxel (taxotere)-resistant patient after treatment with (−)-gossypol acetic acid co-crystals (DP=docetaxel-prednisone regimen; ADP=AT-101 added to DP).

FIG. 22 is a line graph showing PSA response kinetics in a docetaxel (taxotere)-resistant patient after treatment with (−)-gossypol acetic acid co-crystals (DP=docetaxel-prednisone regimen; ADP=AT-101 added to DP).

FIG. 23 is a line graph showing PSA response kinetics in a docetaxel (taxotere)-resistant patient after treatment with (−)-gossypol acetic acid co-crystals (DP=docetaxel-prednisone regimen; ADP=AT-101 added to DP).

FIG. 24 is a bar graph showing a treatment inversion analysis of patients on prior taxotere/prednisone regimen as compared to AT-101/taxotere/prednisone regimen.

FIG. 25 is a bar graph showing a time-to-event plot in taxotere-refractory patients.

### DETAILED DESCRIPTION OF THE INVENTION

The present invention relates to pulsatile dose administration of gossypol. Various compositions comprising gossypol are contemplated for use in the methods of the invention. A gossypol composition may comprise, for example, (±)-gossypol, (−)-gossypol, (+)-gossypol, (±)-gossypol co-crystals, (−)-gossypol co-crystals or (+)-gossypol co-crystals. Gossypol is an inhibitor of anti-apoptotic Bcl-2 family proteins. By inhibiting anti-apoptotic Bcl-2 family proteins, gossypol sensitizes cells to inducers of apoptosis and, in some instances, itself induces apoptosis. Therefore, the invention relates to methods of sensitizing cells to inducers of apoptosis and to methods of inducing apoptosis in cells, comprising administering gossypol by pulsatile dosing alone or in combination with an additional therapeutic agent, such as an inducer of apoptosis. The invention further relates to methods of treating, ameliorating, or preventing disorders in a patient that are responsive to induction of apoptosis comprising administering to the patient gossypol by pulsatile dosing and an inducer of apoptosis, e.g., an anticancer agent. Such disorders include those characterized by a dysregulation of apoptosis and those characterized by overexpression of anti-apoptotic Bcl-2 family proteins. In one embodiment, the disease, condition or disorder responsive to the induction of apoptosis is selected from the group consisting of a hyperproliferative disease, i.e., cancer autoimmune disorder, chronic inflammatory condition, i.e., psoriasis, viral infection, microbial infection, parasitic infection, osteoarthritis, and atherosclerosis.

In one embodiment, the cancer is selected from the group consisting of breast cancer, prostate cancer, lymphoma, skin cancer, pancreatic cancer, colon cancer, malignant melanoma, ovarian cancer, brain cancer, primary brain carcinoma, head-neck cancer, glioma, glioblastoma, liver cancer, bladder cancer, non-small cell lung cancer, head carcinoma, neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, retinoblastoma, neuroendocrine carcinoma, laryngeal cancer, and colorectal cancer. In another embodiment, the cancer is selected from the group consisting of prostate cancer, breast cancer, non-small cell lung cancer, neuroendocrine carcinoma, ovarian carcinoma, Kaposi's sarcoma, myeloma, chronic lymphocytic leukemia, laryngeal cancer, head-neck cancer, colorectal cancer. In another embodiment, the cancer is prostate cancer. In another embodiment, the cancer is non-small cell lung cancer (NSCLC). In another embodiment, the cancer is neuroendocrine carcinoma.

In one embodiment, the cancer is a squamous cell cancer such as squamous cell skin cancer, squamous cell aerodigestive cancer (e.g., NSCLC, laryngeal, nasopharyngeal, tongue, esophagus, stomach, and anal squamous cell cancers), or squamous cell genitourinary cancer (e.g., bladder, penile, cervical, endometrium, and urethra squamous cell cancers). Without intending to be bound by theory, certain types of cancers such as squamous cell cancers, may upregulate particular Bcl-2 protein family members. In addition, these cancers may have particular sensitivity to the induction of proapoptotic Bcl-2 family proteins that is known to occur following gossypol administration. These characteristics may render these cancers, particularly amenable to treatment with gossypol either alone or in combination with other anticancer agents.

The utility of pulsatile dose administration of gossypol in the treatment of squamous cell cancer, e.g., squamous cell non-small cell lung cancer, either alone or in combination with one or more additional anticancer agents in non-trivial. Certain anticancer agents such as bevacizumab and oral vascular endothelial growth factor type 2 receptor tyrosine kinase inhibitors (e.g., sorafenib and motesanib) have excess toxicity and/or higher mortality in patients with squamous cell cancers. For example, there is a black box warning on the bevacizumab label noting contradiction for patients with squamous cell cancers due to bleeding risk. Also, agents such as pemetrexed disodium for injection in combination with cisplatin therapy is approved for the treatment of locally-advanced and metastatic non-small cell lung cancer, and a subgroup analysis of the trials that support pemetrexed disodium use in combination with cisplatin in NSCLC suggest greatest benefit in patients with adenocarcinoma. Thus, there exists a need for safe and efficacious treatment options for patients with squamous cell cancers. The present invention directed to pulsatile dosing of gossypol fills this need.

The term “gossypol,” as used herein refers to (±)-gossypol, (−)-gossypol, or (+)-gossypol, solvates, hydrates, crystalline forms, amorphous forms, co-crystalline forms, and pharmaceutically acceptable salts thereof, unless otherwise indicated.

The term “(−)-gossypol,” as used herein, refers to an optically active composition of gossypol wherein the active molecules comprising the composition rotate plane polarized light counterclockwise (e.g., levorotatory molecules) as measured by a polarimeter. Preferably, the (−)-gossypol compound has an enantiomeric excess of 1% to 100%. In one embodiment, the (−)-gossypol compound has an enantiomeric excess of at least 10%, 20%, 30%, 40%, 50%, 60%, 70%, 80%, 90%, 91%, 92%, 93%, 94%, 95%, 96%, 97%, 98%, 99%, or 100% (−)-gossypol. In one example of a “(−)-gossypol compound”, the specific rotation ([α]_{D}) of the compound is about −350° to about −390°, about −375° to about −390°, or about −385° to about −390°. (See e.g., Dowd, Chirality, 15:486 (2003); Ciesielska et al., Chem. Phys. Lett. 353:69 (2992); Freedman et al., Chirality, 15:196 (2003); and Zhou et al., Kexue Tongbao, 28:1574 (1983)). Methods for resolving racemic gossypol compounds into substantially purified (+)- or (−)-gossypol are known (See e.g., Zhou et al., Kexue Tongbao, 28:1574 (1983) (wherein: L-phenylalanine methyl ester was mixed with the aldehyde groups of gossypol to form a Schiff's base with two diastereoisomers which were then resolved on a normal silica flash chromatography column. The filtrate was concentrated, and the residue was purified by chromatography on silica gel eluting with hexanes:EtOAc=3:1 to give two fractions. Acid hydrolysis of the two fractions in 5N HCl:THF (1:5, room temperature, overnight) regenerated the individual gossypol enantiomers, respectively. The first fraction with a higher R_{f} value contained (−)-gossypol, and the second fraction with a lower R_{f} value contained (+)-gossypol. The crude gossypol fractions were extracted into ether from the residue after removing THF from the reaction mixture. The gossypol fractions were then purified by chromatography on silica gel and eluted with hexanes:EtOAc (3:1 ratio) to give optically pure gossypol, with a yield of 30-40% in two steps. The optical rotatory dispersion values for these products were α_{D}=−352° (c=0.65, CHCl₃) for (−)-gossypol, and α_{D}=+341° (c=0.53, CHCl₃)).

The term “C₁₋₈ carboxylic acid,” as used herein, refers to straight-chained or branched, aromatic or non-aromatic, saturated or unsaturated, substituted or unsubstituted C₁₋₈ carboxylic acid, including, but not limited to, formic acid, acetic acid, propionic acid, n-butyric acid, t-butyric acid, n-pentanoic acid, 2-pentanoic acid, n-hexanoic acid, 2-hexanoic acid, n-heptanoic acid, n-octanoic acid, acrylic acid, succinic acid, fumaric acid, malic acid, tartaric acid, citric acid, lactic acid, and benzoic acid.

The term “C₁₋₈ sulfonic acid,” as used herein, refers to straight-chained or branched, aromatic or non-aromatic, saturated or unsaturated, substituted or unsubstituted C₁₋₈ sulfonic acid, including, but not limited to, methanesulfonic acid, ethanesulfonic acid, n-propanesulfonic acid, 2-propanesulfonic acid, n-butanesulfonic acid, n-pentanesulfonic acid n-hexanesulfonic acid, n-heptanesulfonic acid, n-octanesulfonic acid, and benzenesulfonic acid.

The term “C₁₋₁₂ ketone,” as used herein, refers to straight-chained, cyclic or branched, aromatic or non-aromatic, saturated or unsaturated, substituted or unsubstituted C₁₋₁₂ ketone, including, but not limited to, acetone and cyclododecanone.

The term “C₁₋₁₂ diketone,” as used herein, refers to straight-chained or branched, aromatic or non-aromatic, saturated or unsaturated, substituted or unsubstituted C₁₋₁₂ diketone, including, but not limited to, 2,4-pentanedione.

The term “gossypol co-crystal,” as used herein, refers to a composition comprising co-crystals of (±)-gossypol and a C₁₋₈ carboxylic acid, C₁₋₈ sulfonic acid, C₁₋₁₂ ketone, or C₁₋₁₂ diketone. The term “(−)-gossypol co-crystal,” as used herein, refers to a composition comprising (−)-gossypol and acetic acid, (−)-gossypol and acetone, (−)-gossypol and 2,4-pentanedione, or (−)-gossypol and cyclododecanone. In one embodiment, the (−)-gossypol co-crystal is (−)-gossypol acetic acid co-crystal. See U.S. Pat. No. 7,342,046. A method of preparing (−)-gossypol acetic acid co-crystal is described in U.S. 2008/0021110.

The term “pulsatile dose administration,” as used herein, refers to intermittent (i.e., not constant daily) administration of gossypol. Pulsatile dose administration schedules useful in the present invention encompass any discontinuous daily administration regimen that provides a therapeutically effective amount of gossypol to a patient in need thereof. Pulsatile dosing regimens may use equivalent, lower or higher doses of gossypol than typically used in continuous daily dosing regimens. Expected advantages of pulsatile dose administration of gossypol include, but are not limited to, improved safety, increased gossypol exposure, increased efficacy, and increased patient compliance. These expected advantages may be realized when gossypol is administered as a single agent or is administered in combination with one or more anticancer agents. On the days that gossypol is scheduled to be administered, administration of gossypol may occur once a day, twice-a-day (i.e., BID), three times a day, four times a day or more in accordance with an intermittent daily dosing schedule. In one embodiment, gossypol is administered once- or twice-a-day.

The therapeutic utility of drug administration can be offset by the number and severity of adverse events a patient experiences. Pulsatile dosing of gossypol results in the unexpected combination of a reduction in the number and/or severity of clinical adverse events coupled with a maintenance or enhancement in clinical efficacy, as compared to continuous daily dosing. Moreover, the surprising clinical benefits of pulsatile dose administration of gossypol may be even more prominent when combined with other therapeutic agents.

In addition, administration of gossypol in combination with an anticancer agent (e.g., a chemotherapeutic agent or radiation) to a patient results in an unexpected reduction in the number and/or severity of clinical adverse events experienced by the patient as compared to administration of the anticancer agent in the absence of gossypol. Without intending to be bound by theory, it is possible that gossypol causes cell cycle arrest in normal cells, thereby protecting them from the effects of chemotherapy or radiotherapy. Thus, in one aspect, the invention relates to administration of gossypol to a patient undergoing cancer therapy in order to reduce the number of one or more adverse events, the severity of one or more adverse events, or combination thereof in the patient. According to this aspect, gossypol can be administered by any means (e.g., constant daily or pulsatile dosing) that results in beneficial clinical outcomes. In a particular embodiment, (−)-gossypol is administered according to a pulsatile dosing schedule. In another embodiment, the cancer therapy comprises administration of a chemotherapeutic agent, such as but not limited to, docetaxel. In another embodiment, the cancer therapy comprises administration of radiation. In another embodiment the adverse event(s) are selected from the group consisting of abdominal discomfort, abdominal distention, abdominal pain, albumin decrease, alkaline phosphatase increase, alopecia, ALT increase, anemia, anorexia or decreased appetite, arthralgia, AST increase, asthenia, back pain, constipation, cough, creatinine increase, creatinine phosphokinase increase, decreased appetite, dehydration, diarrhea, dizziness, dry mouth, dysgeusia, dyspnea, fatigue, flatulence, headache, hepatic encephalopathy, hyperbilirubinaemia, hyperglycemia, hyperkalemia, hypocalcemia, hypokalemia, hyponatremia, infection, insomnia, mental status changes, nausea, neutropenia, non cardiac chest pain, pain, pancreatitis, peripheral edema, peripheral sensory neuropathy, pleural effusion, pneumatosis intestinalis, pneumonia, prolongation of QTc, proteinurea, pyrexia, rash, renal failure, sinusitis, small intestinal obstruction (ileus), troponin elevation, troponin I or T increase, urinary tract infection, vomiting, weight decrease, and white blood cells increase. In a particular embodiment, the adverse event(s) are selected from the group consisting of fatigue, neutropenia, anorexia, and peripheral sensory neuropathy.

In one example, gossypol may be administered no more frequently than one day out of every two days (i.e., day 1, day 3, day 5, day 7, day 9, etc.), every three days (i.e., day 1, day 4, day 7, day 10, etc.), every four days, every five days, every six days, every seven days, every eight days, every nine days, every ten days, every two weeks, every three weeks, every four weeks, or longer. The administration of gossypol can continue for one, two, three or four weeks, one, two, three or four months, one, two, three or four years or longer.

In another example, gossypol may be administered on a least two consecutive days, e.g., at least three, four, five, six or seven consecutive days, followed by at least one day, at least two consecutive days, at least three consecutive days, at least four consecutive days, at least five consecutive days, at least six consecutive days, at least seven consecutive days, at least eight consecutive days, at least nine consecutive days, at least ten consecutive days, at least eleven consecutive days, at least twelve consecutive days, at least thirteen consecutive days, at least two consecutive weeks, at least three consecutive weeks or at least four consecutive weeks or longer wherein the gossypol is not administered. The administration of gossypol can continue for one, two, three or four weeks, one, two, three or four months, one, two, three or four years or longer.

In another example, gossypol may be administered twice-a-day for at least three consecutive days followed by eleven, twelve, thirteen, fourteen, fifteen, sixteen, seventeen, eighteen, nineteen or twenty consecutive days, or longer, wherein the gossypol is not administered. In a particular example, gossypol may be administered twice-a-day for three consecutive days followed by eleven consecutive days wherein gossypol is not administered (i.e., gossypol is administered on day 1, day 2, and day 3 of a 14 day treatment cycle). In another particular example, a pharmaceutical composition comprising gossypol (e.g., (−)-gossypol) may be administered twice-a-day for three consecutive days followed by seventeen or eighteen consecutive days wherein gossypol is not administered. The administration of gossypol can continue for two, three or four weeks, one, two, three or four months, one, two, three or four years or longer.

In another example, gossypol (e.g., (−)-gossypol) is administered only on day 1, day 2, and day 3 of a treatment cycle and one or more anticancer agents are administered at least on day 1 of the treatment cycle.

In another example, gossypol (e.g., (−)-gossypol) is administered only on day 1, day 2, day 3, day 4, and day 5 of a treatment cycle and one or more anticancer agents are administered at least on day 1 of the treatment cycle.

Typically, the length of the treatment cycle is determined in accord with the approved dosing protocol(s) of the one or more anticancer agents that are to be administered to the patient in combination with gossypol. In one embodiment, the treatment cycle is about 14 days, about 21 days, or about 28 days. In a particular embodiment, the treatment cycle is 21 days.

In one embodiment, the treatment cycle is repeated one or more times, e.g., 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more times.

In another example, gossypol (e.g., (−)-gossypol) is administered only on day 1, day 2, day 3 of a treatment cycle and one or more anticancer agents are administered starting on day 1 of the treatment cycle in accord with the recommended dosing schedule of the anticancer agent. In one embodiment, the anticancer agent is a chemotherapeutic agent. In another embodiment, the anticancer agent is radiation therapy.

In another example, gossypol (e.g., (−)-gossypol) is administered only on day 1, day 2, day 3, day 4, and day 5 of a treatment cycle and one or more anticancer agents are administered starting on day 1 of the treatment cycle in accord with the recommended dosing schedule of the anticancer agent. In one embodiment, the anticancer agent is a chemotherapeutic agent. In another embodiment, the anticancer agent is radiation therapy.

In another example, gossypol may be administered one day a week, e.g., gossypol administered on one day followed by six consecutive days wherein gossypol is not administered, for at least two weeks, at least three weeks, at least four weeks, at least five weeks, at least six weeks, at least seven weeks or at least eight weeks. The administration of gossypol can continue for one, two, three or four months, one, two, three or four years or longer.

In another example, gossypol may be administered via the sequential use of a combination of two or more pulsatile dosing schedules. The combination may comprise the same pulsatile dosing schedules, e.g., gossypol administered twice-a-day for three consecutive days followed by eleven consecutive days wherein gossypol is not administered followed by gossypol administered twice-a-day for three consecutive days followed by eleven consecutive days wherein gossypol is not administered, or different pulsatile dosing schedules, e.g., gossypol administered twice-a-day for three consecutive days followed by eleven consecutive days wherein gossypol is not administered followed by gossypol administered on one day followed by six consecutive days wherein gossypol is not administered. In one example, pulsatile dose administration of gossypol comprises administration on one day followed by six consecutive days wherein gossypol is not administered followed by administration on at least two consecutive days followed by at least one day wherein gossypol is not administered. In another example, pulsatile dose administration of gossypol comprises administration twice-a-day for at least three consecutive days followed by at least seven consecutive days wherein gossypol is not administered followed by administration on one day followed by at least six consecutive days wherein gossypol is not administered. In another example, pulsatile dose administration of gossypol comprises administration on one day followed by six consecutive days wherein gossypol is not administered followed by administration on one day followed by one day wherein gossypol is not administered. The sequential use of a combination of two or more pulsatile dosing regimens may be repeated as many times as necessary to achieve or maintain a therapeutic response, e.g., from one to about fifty times, e.g., from one to about twenty times, e.g., from about one to about ten times. With every repetition any additional therapeutic agents may be the same or different from that used in the previous repetition.

In another embodiment of the invention, gossypol may be administered according to a pulsatile dosing schedule and/or sequential combination of two or more pulsatile dosing schedules followed by a waiting period. The term “waiting period,” as used herein, refers to a period of time between dosing schedules when gossypol is not administered to the patient. The waiting period may be one, two, three, four, five or six days, one, two or three weeks, one, two, three or four months, one, two, three or four years or longer. In certain embodiments, the waiting period may be one to thirty days, e.g., seven, fourteen, twenty one or thirty days, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. After the waiting period, the same or a different pulsatile dosing schedule and/or sequential combination of one or more pulsatile dosing schedules of gossypol may resume. In one example, pulsatile dose administration of gossypol comprises administration on one day followed by six consecutive days wherein gossypol is not administered, a waiting period and administration on at least two consecutive days followed by at least one day wherein gossypol is not administered. In another example, pulsatile administration of gossypol comprises administration on one day followed by six consecutive days wherein gossypol is not administered, a waiting period and administration on one day followed by six consecutive days wherein gossypol is not administered. In another example, pulsatile dose administration of gossypol comprises administration twice-a-day for at least three consecutive days followed by at least seven consecutive days wherein gossypol is not administered, a waiting period and administration on one day followed by at least six consecutive days wherein gossypol is not administered. The pulsatile dosing/waiting period regimen may be repeated as many times as necessary to achieve or maintain a therapeutic response, e.g., from one to about fifty times, e.g., from one to about twenty times, e.g., from about one to about ten times. With every repetition any additional therapeutic agents may be the same or different from that used in the previous repetition.

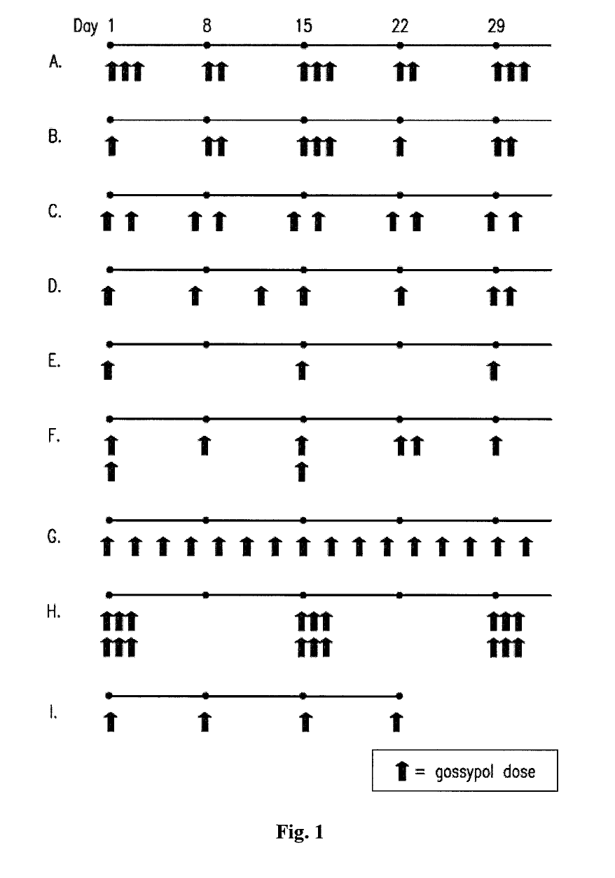

Illustrative pulsatile dose administration schedules are shown in FIG. 1. In addition, referring to FIG. 1, gossypol may be administered, for example, via the sequential use of a combination of schedule A and B, schedule B and C, schedule C and D, schedule D and E, schedule F and G, schedule G and H, schedule H and I, schedule H and H, schedule I and I, schedule H, I and H or schedule G, H and I. Also, gossypol may be administered via schedule H followed by a waiting period (e.g., one to thirty days) followed by administration via schedule I, schedule A-waiting period-schedule B, schedule H-waiting period-schedule E-waiting period-schedule A, schedule H-waiting period-schedule I-waiting period-schedule H or schedule H-schedule I-waiting period-schedule H-schedule I. The above-described pulsatile dose administration schedules are provided for illustrative purposes only and should not be considered limiting. The present invention contemplates any discontinuous daily administration regimen that provides a therapeutically effective amount of gossypol to a patient in need thereof.

The term “adverse event,” as used herein, refers to any undesirable change in health that occurs to a patient during a clinical trial or within a period of time after the clinical trial is complete. Adverse events are categorized by grade, with less serious adverse events given grades 1 (mild) and 2 (moderate) and more serious adverse events given grades 3 (severe) and 4 (life-threatening or disabling). The grading of adverse events can be done using any scale known in the art, such as the National Cancer Institute scale (Common Terminology Criteria for Adverse Events, v3.0). A decrease in the number of adverse events refers to a decrease in the actual number of events. A decrease in the severity of adverse events refers to a decrease in the grade of the adverse events that occur.

The term “Bcl-2 family proteins,” as used herein, refers to both the anti-apoptotic members of the Bcl-2 family, including, but not limited to, Bcl-2, Bcl-XL, Mcl-1, A1/BFL-1, BOO-DIVA, Bcl-w, Bcl-6, Bcl-8, and Bcl-y, and the pro-apoptotic members of the Bcl-2 family, including, but not limited to, Bak, Bax, Bad, tBid, Hrk, Bim, Bmf, as well as other Bcl-2 homology domain 3 (BH3) containing proteins that are regulated by gossypol compounds.

The term “overexpression of anti-apoptotic Bcl-2 family proteins,” as used herein, refers to an elevated level (e.g., aberrant level) of mRNAs encoding for an anti-apoptotic Bcl-2 family protein(s), and/or to elevated levels of anti-apoptotic Bcl-2 family protein(s) in cells as compared to similar corresponding non-pathological cells expressing basal levels of mRNAs encoding anti-apoptotic Bcl-2 family proteins or having basal levels of anti-apoptotic Bcl-2 family proteins. Methods for detecting the levels of mRNAs encoding anti-apoptotic Bcl-2 family proteins or levels of anti-apoptotic Bcl-2 family proteins in a cell include, but are not limited to, Western blotting using anti-apoptotic Bcl-2 family protein antibodies, immunohistochemical methods, and methods of nucleic acid amplification or direct RNA detection. As important as the absolute level of anti-apoptotic Bcl-2 family proteins in cells is to determining that they overexpress anti-apoptotic Bcl-2 family proteins, so also is the relative level of anti-apoptotic Bcl-2 family proteins to other pro-apoptotic signaling molecules (e.g., pro-apoptotic Bcl-2 family proteins) within such cells. When the balance of these two are such that, were it not for the levels of the anti-apoptotic Bcl-2 family proteins, the pro-apoptotic signaling molecules would be sufficient to cause the cells to execute the apoptosis program and die, the cells would be dependent on the anti-apoptotic Bcl-2 family proteins for their survival. In such cells, exposure to an inhibiting effective amount of an anti-apoptotic Bcl-2 family protein inhibitor will be sufficient to cause the cells to execute the apoptosis program and die. Thus, the term “overexpression of an anti-apoptotic Bcl-2 family protein” also refers to cells that, due to the relative levels of pro-apoptotic signals and anti-apoptotic signals, undergo apoptosis in response to inhibiting effective amounts of compounds that inhibit the function of anti-apoptotic Bcl-2 family proteins.

The term “therapeutic agent,” as used herein, refers to any agent which can be used in the treatment, management, or amelioration of a disease, condition or disorder or one or more symptoms thereof. In certain embodiments, the term “therapeutic agent” refers to gossypol, e.g., (−)-gossypol. In certain other embodiments, the term “therapeutic agent” does not refer to gossypol. Preferably, a therapeutic agent is an agent which is known to be useful for, or has been or is currently being used for the treatment, management, prevention, or amelioration of a disorder or one or more symptoms thereof. In one embodiment, the therapeutic agent is an anticancer agent. In one embodiment, the therapeutic agent is one that is used in a premedication regimen.

The terms “anticancer agent” and “anticancer drug” as used herein, refer to any therapeutic agent (e.g., chemotherapeutic compounds and/or molecular therapeutic compounds), radiation therapies, or surgical interventions, used in the treatment of hyperproliferative diseases such as cancer (e.g., in mammals).

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, a therapeutically effective amount preferably refers to the amount of a therapeutic agent that decreases the rate of tumor growth, decreases tumor mass, decreases the number of metastases, increases time to tumor progression, or increases survival time by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The terms “sensitize” and “sensitizing,” as used herein, refer to making, through the administration of gossypol, a patient or a cell within a patient more susceptible, or more responsive, to the biological effects (e.g., promotion or retardation of an aspect of cellular function including, but not limited to, cell growth, proliferation, invasion, angiogenesis, or apoptosis) of a second therapeutic agent. The sensitizing effect of gossypol on a target cell can be measured as the difference in the intended biological effect (e.g., promotion or retardation of an aspect of cellular function including, but not limited to, cell growth, proliferation, invasion, angiogenesis, or apoptosis) observed upon the administration of a second therapeutic agent with and without administration of gossypol. The response of the sensitized cell can be increased by at least 5%, at least 10%, at least 20%, at least 30%, at least 40%, at least 50%, at least 60%, at least 70%, at least 80%, at least 90%, at least 100%, at least 150%, at least 200%, at least 350%, at least 300%, at least 350%, at least 400%, at least 450%, or at least 500% over the response in the absence of gossypol.

The term “dysregulation of apoptosis,” as used herein, refers to any aberration in the ability of (e.g., predisposition) a cell to undergo cell death via apoptosis. Dysregulation of apoptosis is associated with or induced by a variety of conditions, including for example, autoimmune disorders (e.g., systemic lupus erythematosus, rheumatoid arthritis, graft-versus-host disease, myasthenia gravis, or Sjögren's syndrome), chronic inflammatory conditions (e.g., psoriasis, asthma or Crohn's disease), hyperproliferative disorders (e.g., tumors, B cell lymphomas, or T cell lymphomas), viral infections (e.g., herpes, papilloma, or HIV), microbial infections, parasitic infections and other conditions such as osteoarthritis and atherosclerosis. It should be noted that when the dysregulation is induced by or associated with a viral infection, the viral infection may or may not be detectable at the time dysregulation occurs or is observed. That is, viral-induced dysregulation can occur even after the disappearance of symptoms of viral infection.

The term “hyperproliferative disease,” as used herein, refers to any condition in which a localized population of proliferating cells in an animal is not governed by the usual limitations of normal growth, such as cancer. Examples of hyperproliferative disorders include psoriasis, restenosis, tumors, neoplasms, lymphomas and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The term “cancer,” as used herein, is intended to refer to any known cancer, and may include, but is not limited to the following: leukemias such as acute leukemia, acute lymphocytic leukemia, acute myelocytic leukemias such as myeloblastic, promyelocytic, myelomonocytic, monocytic, and erythroleukemia leukemias, and myelodysplastic syndrome; chronic leukemias such as chronic myelocytic (granulocytic) leukemia, chronic lymphocytic leukemia, and hairy cell leukemia; polycythemia vera; lymphomas such as Hodgkin's disease and non-Hodgkin's disease; multiple myelomas such as smoldering multiple myeloma, non-secretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, and synovial sarcoma; brain tumors such as glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancers such as adenocarcinoma, lobular (small cell) carcinoma, intraductal carcinoma, medullary breast cancer, mucinous breast cancer, tubular breast cancer, papillary breast cancer, Paget's disease of the breast, and inflammatory breast cancer; adrenal cancers such as pheochromocytoma and adrenocortical carcinoma; thyroid cancers such as papillary or follicular thyroid cancer, medullary thyroid cancer and anaplastic thyroid cancer; pancreatic cancers such as insulinoma, gastrinoma, glucagonoma, vipoma, somatostatin-secreting tumor, and carcinoid or islet cell tumor; pituitary cancers such as prolactin-secreting tumor and acromegaly; eye cancers such as ocular melanoma, iris melanoma, choroidal melanoma, and cilliary body melanoma, and retinoblastoma; vaginal cancers such as squamous cell carcinoma, adenocarcinoma, and melanoma; vulvar cancers such as squamous cell carcinoma, melanoma, adenocarcinoma, basal cell carcinoma, sarcoma, and Paget's disease of the genitals; cervical cancers such as squamous cell carcinoma and adenocarcinoma; uterine cancers such as endometrial carcinoma and uterine sarcoma; ovarian cancers such as ovarian epithelial carcinoma, ovarian epithelial borderline tumor, germ cell tumor, and stromal tumor; esophageal cancers such as squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as adenocarcinoma, fungaling (polypoid), ulcerating, superficial spreading, diffusely spreading, malignant lymphoma, liposarcoma, fibrosarcoma, and carcinosarcoma; colon cancers; rectal cancers; liver cancers such as hepatocellular carcinoma and hepatoblastoma, gallbladder cancers such as adenocarcinoma; cholangiocarcinomas such as papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as germinal tumor, seminoma, anaplastic, classic (typical), spermatocytic, nonseminoma, embryonal carcinoma, teratoma carcinoma, and choriocarcinoma (yolk-sac tumor), prostate cancers such as adenocarcinoma, leiomyosarcoma, and rhabdomyosarcoma; penile cancers; oral cancers such as squamous cell carcinoma; basal cancers; salivary gland cancers such as adenocarcinoma, mucoepidermoid carcinoma, and adenoidcystic carcinoma; pharynx cancers such as squamous cell cancer and verrucous; skin cancers such as basal cell carcinoma, squamous cell carcinoma and melanoma, superficial spreading melanoma, nodular melanoma, lentigo malignant melanoma, acral lentiginous melanoma; head and neck cancers; kidney cancers such as renal cell cancer, adenocarcinoma, hypernephroma, fibrosarcoma, transitional cell cancer (renal pelvis and/or ureter); Wilms' tumor; and bladder cancers such as transitional cell carcinoma, squamous cell cancer, adenocarcinoma, and carcinosarcoma. In addition, cancers that can be treated by the methods and compositions of the present invention include myxosarcoma, osteogenic sarcoma, endothelio sarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinoma. See Fishman et al., 1985, Medicine, 2d Ed., J.B. Lippincott Co., Philadelphia, Pa. and Murphy et al., 1997, Informed Decisions: The Complete Book of Cancer Diagnosis, Treatment, and Recovery, Viking Penguin, New York, N.Y., for a review of such disorders.

The pathological growth of activated lymphoid cells often results in an autoimmune disorder or a chronic inflammatory condition. As used herein, the term “autoimmune disorder” refers to any condition in which an organism produces antibodies or immune cells which recognize the organism's own molecules, cells or tissues. Non-limiting examples of autoimmune disorders include autoimmune hemolytic anemia, autoimmune hepatitis, Berger's disease or IgA nephropathy, celiac sprue, chronic fatigue syndrome, Crohn's disease, dermatomyositis, fibromyalgia, graft versus host disease, Grave's disease, Hashimoto's thyroiditis, idiopathic thrombocytopenia purpura, lichen planus, multiple sclerosis, myasthenia gravis, psoriasis, rheumatic fever, rheumatic arthritis, scleroderma, Sjögren's syndrome, systemic lupus erythematosus, type 1 diabetes, ulcerative colitis, vitiligo, and the like.

The term “neoplastic disease,” as used herein, refers to any abnormal growth of cells being either benign (non-cancerous) or malignant (cancerous).

The term “anti-neoplastic agent,” as used herein, refers to any compound that retards the proliferation, growth, or spread of a targeted (e.g., malignant) neoplasm.

The terms “prevent,” “preventing” and “prevention,” as used herein, refer to a decrease in the occurrence of pathological cells (e.g., hyperproliferative or neoplastic cells) in an animal. The prevention may be complete, e.g., the total absence of pathological cells in a subject. The prevention may also be partial, such that the occurrence of pathological cells in a subject is less than that which would have occurred without the present invention.

The term “synergistic,” as used herein, refers to an effect obtained when gossypol and a second therapeutic agent are administered together (e.g., at the same time or one after the other) that is greater than the additive effect of gossypol and the second therapeutic agent when administered individually. The synergistic effect allows for lower doses of gossypol and/or the second therapeutic agent to be administered or provides greater efficacy at the same doses. The synergistic effect obtained can be at least 5%, at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 350%, at least 400%, or at least 500% more than the additive effect of gossypol and the second therapeutic agent when administered individually. For example, with respect to the treatment of cancer, the synergistic effect can be a decrease in the rate of tumor growth, a decrease in tumor mass, a decrease in the number of metastases, an increase in time to tumor progression, or an increase in survival time. The co-administration of gossypol by pulsatile dosing and an anticancer agent may allow for the use of lower doses of gossypol and/or the anticancer agent such that the cancer is effectively treated while avoiding any substantial toxicity to the subject.

The term “about,” as used herein, includes the recited number +/−10%. Thus, “about 0.5” means 0.45 to 0.55.

The term “premedication regimen” as used herein refers to administration of one or more therapeutic agents (e.g., corticosteroids, antihistamines) prior to the administration of one or more anticancer agents to a patient in order to minimize the severity of, reduce the number of, or eliminate unwanted side effects (e.g., hypersensitivity reactions) associated with the administration of the anticancer agent(s). Exemplary therapeutic agents that may be used in a premedication regimen include prednisone, dexamethasone, diphenhydramine, cimetidine or ranitidine, or combinations thereof. Additional therapeutic agents used in premedication regimens are known to clinical practioners of ordinary skill in the art. A premedication regimen can be initiated, for example, about 15 minutes, about 30 minutes, about 1 hour, about 3 hours, about 6 hours, about 12 hours, about 1 day, about 2 days, or about 3 days prior to administration of the anticancer agent(s), or on the same day of administration of the anticancer agent(s), and may continue after administration of the anticancer agent(s). In certain embodiments, the methods of the invention comprise a premedication regimen.

Inhibitors of anti-apoptotic Bcl-2 family proteins useful in the present invention include, for example, gossypol or co-crystals of gossypol, or pharmaceutical compositions thereof that are administered by pulsatile dosing. Those skilled in the art will appreciate the importance of compound stability in the manufacturing, storage, shipping, and/or handling of pharmaceutical compositions. Compositions comprising co-crystals of gossypol may be more stable than some other compositions comprising gossypol. Any C₁₋₈ carboxylic acid or C₁₋₈ sulfonic acid that is capable of stabilizing gossypol may be used in the composition. The molar ratio of gossypol to carboxylic acid or sulfonic acid in gossypol co-crystal ranges from about 10:1 to about 1:10, e.g., about 2:1 to about 1:2, e.g., about 1:1.

Compositions comprising gossypol useful in this invention may be prepared using methods known to those of skill in the art, such as the methods disclosed in U.S. Published Application No. 2005/0234135. For example, (−)-gossypol co-crystal may be prepared by dissolving (−)-gossypol in acetone to form a solution, filtering the solution, adding acetic acid into the solution with mixing until the solution turns turbid, leaving the turbid solution at room temperature and then at reduced temperature to form co-crystals, collecting the co-crystals, washing the co-crystals with a solvent, and drying the co-crystals. Reduced temperature is less than about 20° C., preferably about 0-15° C., more preferably about 4° C. The time for co-crystal formation may range from 1 hour to 1 day; preferably the time is about 1-4 hours. The co-crystals may be collected by any suitable means, including by filtration. The solvent for washing the co-crystals may be any suitable solvent, e.g., hexane, pentane, benzene, toluene, or petroleum ether. The washed co-crystals may be dried at room temperature, preferably in a lightproof container. The co-crystals may also be dried in a vacuum drier, preferably at an elevated temperature (e.g., about 30-60° C., more preferably about 40° C.) for about 6-72 hours, preferably about 12-48 hours.

Gossypol acetic acid co-crystals may also be prepared from a mixture of (±)-gossypol and acetic acid via recrystallization. The gossypol acetic acid co-crystals may be further recrystallized from a solution of gossypol acetic acid in a mixture of acetone and acetic acid. The recrystallization mixture is held for about 15 minutes to about 100 minutes, e.g., about 30 minutes to about 60 minutes, to allow co-crystal formation. The recrystallization is carried out at ambient temperature, e.g., about 15° C. to about 30° C., e.g., about 22° C. Following the recrystallization, the crystals are harvested from the recrystallization mixture (e.g., by filtration) and washed with a non-polar solvent, e.g., pentane, hexene, hexane(s), heptane, or mixtures thereof. Preferably, the washing step is quick to avoid the incorporation of the non-polar solvent into the crystals. Short washing times (less than 2 minutes) are preferred. The crystals may then be dried, e.g., in vacuo, while protected from light. The recrystallization may be repeated more than once (e.g., 2, 3, 4, 5, or more times) to improve the impurity profile, e.g., until gossypol co-crystals comprise less than about 5%, 4%, 3%, 2%, 1%, 0.5%, or 0.1% impurities. Once the desired impurity profile is obtained, a final recrystallization may be carried out at a lower temperature, e.g., about −20° C. to about 10° C., preferably about −10° C. to about 0° C. The recrystallization mixture is held for about 15 minutes to about 100 minutes, e.g., about 30 to about 60 minutes, and the resulting crystals are then filtered, washed and dried as described above. This method of producing gossypol acetic acid co-crystals produces a final product comprising less than about 5% total impurities, preferably less than about 3%, 2%, or 1%.

(−)-Gossypol acetic acid co-crystals may also be prepared from gossypol acetic acid co crystals via imine (Schiff base) formation with an optically active amine to form diastereomers. As used herein, the term “imine” includes other tautomers such as eneamine tautomers and stereoisomers thereof. The optically active amine may be, but is not limited to, L-phenylalanine methyl ester, S-1-methylphenethylamine, or L-phenylalaminol or the corresponding HCl salt. The derivatization may be carried out in the absence of oxygen, e.g., under a nitrogen purge. The derivatization is carried out in the presence of a nonpolar and/or polar solvent, e.g., dichloromethane and/or isopropanol, for a time period of about 0.5 to about 3 hours, e.g., about 1 hour to about 2 hours. A dehydrating agent such as sodium sulfate or a molecular sieve, e.g., type 3 Å, is then added, along with suitable reagents for buffering the reaction mix at a pH of about 5 to about 7, e.g., about 6. One suitable buffering agent is sodium bicarbonate. The reaction mixture is then stirred for at least about 15 minutes, e.g., at least about 30 minutes. The progression of the reaction may be monitored for completion. For example, the reaction mixture may be assayed for the absence of gossypol using thin layer chromatography (TLC) or preferably in real time by high pressure liquid chromatography (HPLC). If the reaction is incomplete, the pH may be adjusted back to about 6 by adding further buffering agents. The reaction is then continued for about 24 hours and again checked for completion of the reaction. After completion, the reaction mixture may be filtered to remove the solids and the solids washed with additional non-polar solvent, e.g., dichloromethane. The filtrates may then be evaporated to dryness, e.g., with a rotary evaporator with the bath set at about 30° C. to about 40° C.

The resultant diastereomers are then separated, e.g., by chromatography For example, the diastereomers are separated by silica gel chromatography, e.g., Kromasil Si. The dried filtrate is reconstituted in a non-polar solvent, e.g., dichloromethane, and charged onto the column. The diastereomers are eluted with a solvent system comprising non-polar and polar solvents, e.g., 1:1 heptane:ethyl acetate. Column fractions may be monitored by HPLC and fractions containing the desired isomer (e.g., at least about 90%) may be pooled and evaporated. Impure fractions may be collected and passed over the column additional times. In one embodiment, the diastereomer mixture is held for less than 24 hours, preferably less than 12, 6, or 3 hours, prior to separation in order to avoid any increase in contamination with gossypol derivatives.

The separated R-(−)-gossypol bis-imine diastereomer may then be hydrolyzed to produce (−)-gossypol. The hydrolysis may be carried out in the absence of oxygen, e.g., under a nitrogen purge. The (−)-gossypol derivative is mixed with a polar solvent (e.g., tetrahydrofuran) and an acid (e.g., aqueous hydrochloric acid) and stirred for at least about 1 hour, e.g., at least about 5 hours. The extent of the reaction may be monitored by TLC or HPLC for depletion of both the bis- and mono-imine compounds to less than about 10%, preferably less than about 5%. If the reaction is not sufficient, it may be continued for at least about 15 hours and re-evaluated.

Once sufficient hydrolysis has occurred, the reaction mixture may then be washed with an aqueous brine solution. The aqueous brine solution may be back extracted with a polar solvent (e.g., ethyl acetate). The organic layers are then combined and washed with an alkaline aqueous solution (e.g., sodium bicarbonate) followed by a brine solution. The organic layers may then be evaporated to dryness, e.g., with a rotary evaporator with the bath set at about 30° C. to about 40° C.

The crude isolate is then dissolved in a solvent system comprising non-polar and polar solvents (e.g., 1:1 heptane:ethyl acetate) and passed over a silica gel plug using the same solvent system containing a small amount of acetic acid (to avoid sticking to the plug). Fractions may be collected and monitored for gossypol content using TLC or HPLC. Product-containing fractions may be pooled and evaporated to dryness, e.g., with a rotary evaporator with the bath set at about 30° C. to about 40° C.

If further purification of the (−)-gossypol is desired, the (−)-gossypol may be purified by chromatography over a hydrophilic resin, e.g., a dihydroxypropyl resin such as DIOL, e.g., YMC DIOL (120 angstrom×10-20 micron) (GL Sciences). The dried product from the previous step may be reconstituted in a solvent system comprising non-polar and polar solvents (e.g., 1:1 heptane:ethyl acetate) and purified over the column using the same solvent system. Fractions are collected, held at a reduced temperature (e.g., about 2° C. to about 8° C.), and the fractions assayed for gossypol content using TLC or HPLC. Fractions containing gossypol (e.g., at least 90%) may be pooled and evaporated to dryness, e.g., with a rotary evaporator with the bath set at about 30° C. to about 40° C. Fractions with less than 90% gossypol may be pooled and re-purified over the column.

As a final step, the purified (−)-gossypol may be dissolved in acetone (e.g., at about 4 mL per 1 g gossypol) and glacial acetic acid is added (about 1.5 mL per 1 g gossypol). The mixture may then be loaded into a suitable container for crystallization (e.g., a Büchi Ball). If there is no immediate crystallization, the solvent may be slowly removed by vacuum until a crystal mass appears. The mixture may then be held for about 15 minutes to about 100 minutes, e.g., about 30 minutes to about 60 minutes, and then filtered. The crystals may then be washed with the same ratio of acetone and acetic acid. Finally, the crystals may be soaked in acetic acid (about 3 mL per 1 g gossypol for about 20 to about 40 minutes, preferably about 30 minutes, and the acetic acid removed by filtration. The crystals may then be dried (e.g., in vacuo) for at least one hour, e.g., about 2 to about 4 hours. The crystals may be packaged and stored protected from light (e.g., in amber glass vials) at a reduced temperature (e.g., about −30° C. to about 0° C., preferably about −10° C. to about −20° C.

An alternative method of derivatizing gossypol acetic acid starting material comprising (±)-gossypol to form a Schiff base in the above-described methods of producing (−)-gossypol acetic acid co-crystals comprises treating a mixture of gossypol acetic acid starting material and optically active amine (e.g., L-phenylalanine methyl ester hydrochloride) in a nonpolar solvent (e.g., dichloromethane) with triethylamine and mixing for at least 2 hours, e.g., about 5 hours, optionally under an oxygen-free atmosphere. The reaction may be monitored for completion using HPLC or TLC methods. After completion of the reaction, the mixture is extracted with water and the organic phase separated and evaporated to dryness, e.g., with a rotary evaporator with the bath set at about 25° C. to about 35° C., followed by a high vacuum overnight.

Gossypol been shown to bind to Bcl-2, Bcl-X_{L}, and Mcl-1 at the BH3 binding groove and to have anticancer activity (U.S. Patent Application No. 2003/0008924). Thus, gossypol may be used to induce apoptosis and also potentiate the induction of apoptosis in response to apoptosis induction signals when administered by pulsatile dosing. It is contemplated that gossypol administered by pulsatile dosing will sensitize cells to inducers of apoptosis, including cells that are resistant to such inducers. Gossypol administered by pulsatile dosing can be used to induce apoptosis in any disorder that can be treated, ameliorated, or prevented by the induction of apoptosis. Thus, the present invention provides methods for targeting patients characterized as overexpressing an anti-apoptotic Bcl-2 family protein. In some of the embodiments, the cells (e.g., cancer cells) show elevated expression levels of one or more anti-apoptotic Bcl-2 family proteins as compared to non-pathological samples (e.g., non-cancerous cells). In other embodiments, the cells operationally manifest elevated expression levels of anti-apoptotic Bcl-2 family proteins by virtue of executing the apoptosis program and dying in response to administration of an inhibiting effective amount of gossypol, the response occurring, at least in part, due to the dependence in such cells on anti-apoptotic Bcl-2 family protein function for their survival.

In some embodiments, the methods of the present invention are used to treat diseased cells, tissues, organs, or pathological conditions and/or disease states in a patient (e.g., a mammalian subject including, but not limited to, humans and veterinary animals). In this regard, various diseases and pathologies are amenable to treatment or prophylaxis using the present methods. A non-limiting exemplary list of these diseases and conditions includes, but is not limited to, cancers, T and B cell mediated autoimmune diseases, inflammatory diseases, infections, hyperproliferative diseases, AIDS, degenerative conditions, vascular diseases, and the like. In some embodiments, the cancer cells being treated are metastatic. In other embodiments, the cancer cells being treated are resistant to anticancer agents, e.g., taxanes, e.g., docetaxel.

In some embodiments, infections suitable for treatment with the methods of the present invention include, but are not limited to, infections caused by viruses, bacteria, fungi, parasites, mycoplasma, prions, and the like.

The present invention contemplates that any known therapeutic utility of gossypol may be exploited via pulsatile dose administration of gossypol. In certain embodiments, gossypol administered by pulsatile doing can be used to modulate spermicidal activity (e.g., function as a male contraceptive or antifertility agent), treat malaria, microbial or viral disease (e.g., inhibit the growth of the HIV virus as a treatment for AIDS), treat obesity, skin disorders or baldness, inhibit growth of endothelial cells, inhibit vascularization or neovascularization, treat arthritic conditions, neovascular-based dermatological conditions, diabetic retinopathy, Kaposi's sarcoma, age-related macular degeneration, restenosis, telangiectasia, glaucoma, keloids, corneal graft rejection, wound granularization, angiofibroma, Osler-Webber syndrome, myocardial angiogenesis, psoriatic arthritis or scleroderma, inhibit DNA synthesis or DNA polymerase activity and treat gynecological disorders (e.g., endometriosis) or diabetic complications.

Some embodiments of the present invention provides methods for administering an effective amount of gossypol by pulsatile dosing and at least one additional therapeutic agent (including, but not limited to, chemotherapeutic agents, antineoplastic agents, antimicrobial agents, antiviral agents, antifungal agents, and anti-inflammatory agents) and/or therapeutic technique (e.g., surgical intervention, and/or radiotherapeutic agent). The term “chemotherapeutic agent,” as used herein, refers to any chemical substance known to those of skill in the art to be effective for the treatment or amelioration of cancer and/or as an inducer of apoptosis.

In some embodiments, the combination of gossypol administered by pulsatile dosing and one or more therapeutic agents will have a greater effect as compared to the administration of either gossypol or therapeutic agent alone. In other embodiments, the combination of gossypol administered by pulsatile dosing and one or more therapeutic agents is expected to result in a synergistic effect (i.e., more than additive) as compared to the administration of either one alone. In some embodiments, pulsatile dose administration of gossypol results in a reduction in number and/or decreased severity of adverse events in patients. In some embodiments, the combination of gossypol administered by pulsatile dosing and one or more anticancer agents overcomes patient chemoresistance to the anticancer agent.

In a particular embodiment, the invention pertains to a method of treating, ameliorating or preventing cancer comprising administering to a patient in need thereof (−)-gossypol, wherein about 20 mg to about 60 mg of the (−)-gossypol is administered twice-a-day for three consecutive days followed by about eleven consecutive days wherein the (−)-gossypol is not administered. In one embodiment, the cancer is selected from the group consisting of non-small cell lung cancer and neuroendocrine carcinoma.

In a particular embodiment, the invention pertains to a method of treating, ameliorating or preventing cancer comprising administering to a patient in need thereof (−)-gossypol in combination with an anticancer agent, wherein about 20 mg to about 60 mg of the (−)-gossypol is administered twice-a-day for three consecutive days followed by about eighteen consecutive days wherein the (−)-gossypol is not administered. In one embodiment, the anticancer agent is docetaxel. In another embodiment, the anticancer agent is paclitaxel. In one embodiment, the patient has demonstrated chemoresistance to docetaxel or paclitaxel treatment. In one embodiment, the cancer is prostate cancer. In another embodiment, the cancer is non-small cell lung cancer.

In another particular embodiment, the invention pertains to a method of treating, ameliorating or preventing cancer comprising administering to a patient in need thereof (−)-gossypol in combination with an anticancer agent, wherein about 20 mg to about 60 mg of the (−)-gossypol is administered twice-a-day for five consecutive days followed by about sixteen consecutive days wherein the (−)-gossypol is not administered. In one embodiment, the anticancer agent is topotecan. In one embodiment, the patient has demonstrated chemoresistance to topotecan cancer therapy. In one embodiment, the cancer is non-small cell lung cancer.

In an another particular embodiment, the invention pertains to a method of treating, ameliorating or preventing non-small cell lung cancer comprising administering to a patient in need thereof (−)-gossypol in combination with erlotinib, wherein (−)-gossypol is administered on a pulsatile dosing schedule. In one embodiment, (−)-gossypol is administered on at least three consecutive days followed by at least eleven consecutive days wherein (−)-gossypol is not administered. In one embodiment, the patient has demonstrated chemoresistance to erlotinib cancer therapy.

In another particular embodiment, the invention pertains to a method of treating, ameliorating or preventing multiple myeloma or chronic lymphocytic leukemia comprising administering to a patient in need thereof (−)-gossypol in combination with lenalidomide, wherein (−)-gossypol is administered on a pulsatile dosing schedule. In one embodiment, (−)-gossypol is administered on at least three consecutive days followed by at least eleven consecutive days wherein (−)-gossypol is not administered. In one embodiment, the patient has demonstrated chemoresistance to lenalidomide cancer therapy.

In another particular embodiment, the invention pertains to a method of treating, ameliorating or preventing laryngeal cancer comprising administering to a patient in need thereof (−)-gossypol in combination with cisplatin, wherein (−)-gossypol is administered on a pulsatile dosing schedule. In one embodiment, (−)-gossypol is administered on at least three consecutive days followed by at least eleven consecutive days wherein (−)-gossypol is not administered. In one embodiment, the patient has demonstrated chemoresistance to cisplatin cancer therapy.

In another particular embodiment, the invention pertains to a method of treating, ameliorating or preventing head and neck cancer comprising administering to a patient in need thereof (−)-gossypol in combination with erbitux, wherein (−)-gossypol is administered on a pulsatile dosing schedule. In one embodiment, (−)-gossypol is administered on at least three consecutive days followed by at least eleven consecutive days wherein (−)-gossypol is not administered. In one embodiment, the patient has demonstrated chemoresistance to erbitux cancer therapy.

In another particular embodiment, the invention pertains to a method of treating, ameliorating or preventing colorectal cancer comprising administering to a patient in need thereof (−)-gossypol in combination with oxaliplatin, wherein (−)-gossypol is administered on a pulsatile dosing schedule. In one embodiment, (−)-gossypol is administered on at least three consecutive days followed by at least eleven consecutive days wherein (−)-gossypol is not administered. In one embodiment, the patient has demonstrated chemoresistance to oxaliplatin cancer therapy.

In another particular embodiment, the invention pertains to a method of treating, ameliorating or preventing prostate cancer comprising administering to a subject in need thereof (−)-gossypol only on day 1, day 2, and day 3 of a 21-day treatment cycle, wherein the (−)-gossypol is administered in combination with one or more anticancer agents selected from the group consisting of docetaxel; docetaxel and prednisone; mitoxantrone; and mitoxantrone and prednisone. In one embodiment, at least one anticancer agent is administered at least on day 1 of the 21-day treatment cycle. In a particular embodiment, the anticancer agent is docetaxel and prednisone.

In another particular embodiment, the invention pertains to a method of treating, ameliorating or preventing non-small cell lung cancer comprising administering to a subject in need thereof (−)-gossypol only on day 1, day 2, and day 3 of a 21-day treatment cycle, wherein the (−)-gossypol is administered in combination with one or more anticancer agents selected from the group consisting of docetaxel; cisplatin and etoposide; and erlotinib. In one embodiment, at least one anticancer agent is administered at least on day 1 of the 21-day treatment cycle.

In another particular embodiment, the invention pertains to a method of treating, ameliorating or preventing breast cancer, non-small cell lung cancer, ovarian carcinoma, or Kaposi's sarcoma comprising administering to a subject in need thereof (−)-gossypol only on day 1, day 2, and day 3 of a 21-day or 28-day treatment cycle, wherein the (−)-gossypol is administered in combination with paclitaxel. In one embodiment, paclitaxel is administered at least on day 1 of the 21-day or 28-day treatment cycle.

In another particular embodiment, the invention pertains to a method of treating, ameliorating or preventing multiple myeloma or chronic lymphocytic leukemia comprising administering to a subject in need thereof (−)-gossypol only on day 1, day 2, and day 3 of a 21-day treatment cycle, wherein the (−)-gossypol is administered in combination with lenalidomide. In one embodiment, lenalidomide is administered at least on day 1 of the 21-day treatment cycle.

In another particular embodiment, the invention pertains to a method of treating, ameliorating or preventing head and neck cancer comprising administering to a subject in need thereof (−)-gossypol only on day 1, day 2, and day 3 of a 21-day treatment cycle, wherein the (−)-gossypol is administered in combination with one or more anticancer agents selected from the group consisting of cisplatin; cisplatin and radiation; erbitux; or carboplatin, 5-fluorouracil, and cetuximab. In one embodiment, at least one anticancer agent is administered at least on day 1 of the 21-day treatment cycle.

In another particular embodiment, the invention pertains to a method of treating, ameliorating or preventing follicular lymphoma comprising administering to a subject in need thereof (−)-gossypol only on day 1, day 2, and day 3 of a 21-day treatment cycle, wherein the (−)-gossypol is administered in combination with rituximab, cyclophosphamide, doxorubicin, and vincristine. In one embodiment, at least one of rituximab, cyclophosphamide, doxorubicin, or vincristine is administered at least on day 1 of the 21-day treatment cycle.

In another particular embodiment, the invention pertains to a method of treating, ameliorating or preventing non-small cell lung cancer comprising administering to a subject in need thereof (−)-gossypol only on day 1, day 2, day 3, day 4, and day 5 of a 21-day treatment cycle, wherein the (−)-gossypol is administered in combination with topotecan HCl. In one embodiment, topotecan HCl is administered at least on day 1 of the 21-day treatment cycle.

In another particular embodiment, the invention pertains to a method of treating, ameliorating or preventing glioblastoma multiforme comprising administering to a subject in need thereof (−)-gossypol only on day 1, day 2, and day 3 of a 21-day treatment cycle, wherein the (−)-gossypol is administered in combination with one or more anticancer agents selected from the group consisting of temozolomide and temozolomide and radiation. In one embodiment, at least one anticancer agent is administered at least on day 1 of the 21-day treatment cycle.

A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics (e.g., BH3 mimetics); agents that bind (e.g., oligomerize or complex) with a Bcl-2 family protein such as Bax; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with gossypol or compositions thereof are known to those skilled in the art.

In certain embodiments, anticancer agents comprise agents that induce or stimulate apoptosis. Agents that induce apoptosis include, but are not limited to, radiation (e.g., X-rays, gamma rays, UV); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, BEXXAR, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbuta zone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine, dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine; and ara-C, and the like.

In still other embodiments, the methods of the present invention provide pulsatile dose administration of gossypol and at least one anti-hyperproliferative or antineoplastic agent; e.g., selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil), floxuridine (fluorode-oxyuridine), and cytarabine (cytosine arabinoside)); and 3) purine analogs (e.g., mercaptopurine (6-mercaptopurine), thioguanine (6-thioguanine), and pentostatin (2′-deoxycoformycin)).

In still further embodiments, chemotherapeutic agents suitable for use in the compositions and methods of the present invention include, but are not limited to: 1) vinca alkaloids (e.g., vinblastine, vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin and carboplatin); 7) anthracenediones (e.g., mitoxantrone); 8) substituted ureas (e.g., hydroxyurea); 9) methylhydrazine derivatives (e.g., procarbazine (N-methylhydrazine)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is used in a cancer therapy context finds use in the methods of the present invention. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., |
| (des-alanyl-1, serine-125 human | | Emeryville, CA |
| interleukin-2) | | |
| Alemtuzumab | Campath | Millennium and |
| (IgG1κ anti CD52 antibody) | | ILEX Partners, |
| | | LP, Cambridge, |
| | | MA |
| Alitretinoin | Panretin | Ligand |
| (9-cis-retinoic acid) | | Pharmaceuticals, |
| | | Inc., San Diego |
| | | CA |
| Allopurinol | Zyloprim | GlaxoSmithKline, |
| (1,5-dihydro-4H-pyrazolo[3,4- | | Research Triangle |
| d]pyrimidin-4-one monosodium salt) | | Park, NC |
| Altretamine | Hexalen | US Bioscience, |
| (N,N,N′,N′,N″,N″,-hexamethyl-1,3,5- | | West |
| triazine-2,4,6-triamine) | | Conshohocken, |
| | | PA |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a,a,a′,a′- | | Pharmaceuticals, |
| tetramethyl-5-(1H-1,2,4-triazol-1- | | LP, Wilmington, |
| ylmethyl)) | | DE |
| Arsenic trioxide | Trisenox | Cell Therapeutic, |
| | | Inc., Seattle, WA |
| Asparaginase | Elspar | Merck & Co., |
| (L-asparagine amidohydrolase, type EC-2) | | Inc., Whitehouse |
| | | Station, NJ |
| BCG Live | TICE BCG | Organon Teknika, |
| (lyophilized preparation of an attenuated | | Corp., Durham, |
| strain of Mycobacterium bovis (Bacillus | | NC |
| Calmette-Gukin [BCG], substrain | | |
| Montreal) | | |
| bexarotene capsules | Targretin | Ligand |
| (4-[1-(5,6,7,8-tetrahydro-3,5,5,8,8- | | Pharmaceuticals |
| pentamethyl-2-napthalenyl) ethenyl] | | |
| benzoic acid) | | |
| bexarotene gel | Targretin | Ligand |
| | | Pharmaceuticals |
| Bleomycin | Blenoxane | Bristol-Myers |
| (cytotoxic glycopeptide antibiotics | | Squibb Co., NY, |
| produced by Streptomyces verticillus; | | NY |
| bleomycin A₂ and bleomycin B₂) | | |
| Capecitabine | Xeloda | Roche |
| (5′-deoxy-5-fluoro-N- | | |
| [(pentyloxy)carbonyl]-cytidine) | | |
| Carboplatin | Paraplatin | Bristol-Myers |
| (platinum, diammine [1,1- | | Squibb |
| cyclobutanedicarboxylato(2-)-0,0′]-,(SP- | | |
| 4-2)) | | |
| Carmustine | BCNU, | Bristol-Myers |
| (1,3-bis(2-chloroethyl)-1-nitrosourea) | BiCNU | Squibb |
| Carmustine with Polifeprosan 20 Implant | Gliadel Wafer | Guilford |
| | | Pharmaceuticals, |
| | | Inc., Baltimore, |
| | | MD |
| Celecoxib | Celebrex | Searle |
| (as 4-[5-(4-methylphenyl)-3- | | Pharmaceuticals, |
| (trifluoromethyl)-1H-pyrazol-1-yl] | | England |
| benzenesulfonamide) | | |
| Chlorambucil | Leukeran | GlaxoSmithKline |
| (4- | | |
| [bis(2chlorethyl)amino]benzenebutanoic | | |
| acid) | | |
| Cisplatin | Platinol | Bristol-Myers |
| (PtCl₂H₆N₂) | | Squibb |
| Cladribine | Leustatin, 2- | R. W. Johnson |
| (2-chloro-2′-deoxy-b-D-adenosine) | CdA | Pharmaceutical |
| | | Research Institute, |
| | | Raritan, NJ |
| Cyclophosphamide | Cytoxan, | Bristol-Myers |
| (2-[bis(2-chloroethyl)amino] tetrahydro- | Neosar | Squibb |
| 2H-13,2-oxazaphosphorine 2-oxide | | |
| monohydrate) | | |
| Cytarabine | Cytosar-U | Pharmacia & |
| (1-b-D-Arabinofuranosylcytosine, | | Upjohn Company |
| C₉H₁₃N₃O₅) | | |
| cytarabine liposomal | DepoCyt | Skye |
| | | Pharmaceuticals, |
| | | Inc., San Diego, |
| | | CA |
| Dacarbazine | DTIC-Dome | Bayer AG, |
| (5-(3,3-dimethyl-1-triazeno)-imidazole-4- | | Leverkusen, |
| carboxamide (DTIC)) | | Germany |
| Dactinomycin, actinomycin D | Cosmegen | Merck |
| (actinomycin produced by Streptomyces | | |
| parvullus, C₆₂H₈₆N₁₂O₁₆) | | |
| Darbepoetin alfa | Aranesp | Amgen, Inc., |
| (recombinant peptide) | | Thousand Oaks, |
| | | CA |
| daunorubicin liposomal | DanuoXome | Nexstar |
| ((8S-cis)-8-acetyl-10-[(3-amino-2,3,6- | | Pharmaceuticals, |
| trideoxy-a-L-lyxo-hexopyranosyl)oxy]- | | Inc., Boulder, CO |
| 7,8,9,10-tetrahydro-6,8,11-trihydroxy-1- | | |
| methoxy-5,12-naphthacenedione | | |
| hydrochloride) | | |
| Daunorubicin HCl, daunomycin | Cerubidine | Wyeth Ayerst, |
| ((1S,3S)-3-Acetyl-1,2,3,4,6,11- | | Madison, NJ |
| hexahydro-3,5,12-trihydroxy-10-methoxy- | | |
| 6,11-dioxo-1-naphthacenyl 3-amino-2,3,6- | | |
| trideoxy-(alpha)-L-lyxo-hexopyranoside | | |
| hydrochloride) | | |
| Denileukin diftitox | Ontak | Seragen, Inc., |
| (recombinant peptide) | | Hopkinton, MA |
| Dexrazoxane | Zinecard | Pharmacia & |
| ((S)-4,4′-(1-methyl-1,2-ethanediyl)bis-2,6- | | Upjohn Company |
| piperazinedione) | | |
| Docetaxel | Taxotere | Aventis |
| ((2R,3S)—N-carboxy-3-phenylisoserine, N- | | Pharmaceuticals, |
| tert-butyl ester, 13-ester with 5b-20- | | Inc., Bridgewater, |
| epoxy-12a,4,7b,10b,13a-hexahydroxytax- | | NJ |
| 11-en-9-one 4-acetate 2-benzoate, | | |
| trihydrate) | | |
| Doxorubicin HCl | Adriamycin, | Pharmacia & |
| (8S,10S)-10-[(3-amino-2,3,6-trideoxy-a- | Rubex | Upjohn Company |
| L-lyxo-hexopyranosyl)oxy]-8-glycolyl- | | |
| 7,8,9,10-tetrahydro-6,8,11-trihydroxy-1- | | |
| methoxy-5,12-naphthacenedione | | |
| hydrochloride) | | |
| doxorubicin | Adriamycin | Pharmacia & |
| | PFS | Upjohn Company |
| | Intravenous | |
| | injection | |
| doxorubicin liposomal | Doxil | Sequus |
| | | Pharmaceuticals, |
| | | Inc., Menlo park, |
| | | CA |
| dromostanolone propionate | Dromostanolone | Eli Lilly & |
| (17b-Hydroxy-2a-methyl-5a-androstan-3- | | Company, |
| one propionate) | | Indianapolis, IN |
| dromostanolone propionate | Masterone | Syntex, Corp., |
| | injection | Palo Alto, CA |
| Elliott's B Solution | Elliott's B | Orphan Medical, |
| | Solution | Inc |
| Epirubicin | Ellence | Pharmacia & |
| ((8S-cis)-10-[(3-amino-2,3,6-trideoxy-a- | | Upjohn Company |
| L-arabino-hexopyranosyl)oxy]-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & |
| (estra-1,3,5(10)-triene-3,17-diol(17(beta))-, | | Upjohn Company |
| 3-[bis(2-chloroethyl)carbamate] 17- | | |
| (dihydrogen phosphate), disodium salt, | | |
| monohydrate, or estradiol 3-[bis(2- | | |
| chloroethyl)carbamate] 17-(dihydrogen | | |
| phosphate), disodium salt, monohydrate) | | |
| Etoposide phosphate | Etopophos | Bristol-Myers |
| (4′-Demethylepipodophyllotoxin 9-[4,6-O- | | Squibb |
| (R)-ethylidene-(beta)-D-glucopyranoside], | | |
| 4′-(dihydrogen phosphate)) | | |
| etoposide, VP-16 | Vepesid | Bristol-Myers |
| (4′-demethylepipodophyllotoxin 9-[4,6-0- | | Squibb |
| (R)-ethylidene-(beta)-D-glucopyranoside]) | | |
| Exemestane | Aromasin | Pharmacia & |
| (6-methylenandrosta-1,4-diene-3,17- | | Upjohn Company |
| dione) | | |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex |
| (fluorinated nucleotide analog of the | | Laboratories, Inc., |
| antiviral agent vidarabine, 9-b-D- | | Cedar Knolls, NJ |
| arabinofuranosyladenine (ara-A)) | | |
| Fluorouracil, 5-FU | Adrucil | ICN |
| (5-fluoro-2,4(1H,3H)-pyrimidinedione) | | Pharmaceuticals, |
| | | Inc., Humacao, |
| | | Puerto Rico |
| Fulvestrant | Faslodex | IPR |
| (7-alpha-[9-(4,4,5,5,5-penta | | Pharmaceuticals, |
| fluoropentylsulphinyl) nonyl]estra-1,3,5- | | Guayama, Puerto |
| (10)-triene-3,17-beta-diol) | | Rico |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex | AstraZeneca |
| (acetate salt of [D- | Implant | Pharmaceuticals |
| Ser(But)⁶,Azgly¹⁰]LHRH; pyro-Glu-His- | | |
| Trp-Ser-Tyr-D-Ser(But)-Leu-Arg-Pro- | | |
| Azgly-NH2 acetate [C₅₉H₈₄N₁₈O₁₄•(C₂H₄O₂)ₓ | | |
| Hydroxyurea | Hydrea | Bristol-Myers |
| | | Squibb |
| Ibritumomab Tiuxetan | Zevalin | Biogen IDEC, |
| (immunoconjugate resulting from a | | Inc., Cambridge |
| thiourea covalent bond between the | | MA |
| monoclonal antibody Ibritumomab and the | | |
| linker-chelator tiuxetan [N-[2- | | |
| bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & |
| (5,12-Naphthacenedione, 9-acetyl-7-[(3- | | Upjohn Company |
| amino-2,3,6-trideoxy-(alpha)-L-lyxo- | | |
| hexopyranosyl)oxy]-7,8,9,10-tetrahydro- | | |
| 6,9,11-trihydroxyhydrochloride, (7S-cis)) | | |
| Ifosfamide | IFEX | Bristol-Myers |
| (3-(2-chloroethyl)-2-[(2- | | Squibb |
| chloroethyl)amino]tetrahydro-2H-1,3,2- | | |
| oxazaphosphorine 2-oxide) | | |
| Imatinib Mesilate | Gleevec | Novartis AG, |
| (4-[(4-Methyl-1-piperazinyl)methyl]-N- | | Basel, Switzerland |
| [4-methyl-3-[[4-(3-pyridinyl)-2- | | |
| pyrimidinyl]amino]-phenyl]benzamide | | |
| methanesulfonate) | | |
| Interferon alfa-2a | Roferon-A | Hoffmann-La |
| (recombinant peptide) | | Roche, Inc., |
| | | Nutley, NJ |
| Interferon alfa-2b | Intron A | Schering AG, |
| (recombinant peptide) | (Lyophilized | Berlin, Germany |
| | Betaseron) | |
| Irinotecan HCl | Camptosar | Pharmacia & |
| ((4S)-4,11-diethyl-4-hydroxy-9-[(4- | | Upjohn Company |
| piperi-dinopiperidino)carbonyloxy]-1H- | | |
| pyrano[3′,4′:6,7] indolizino[1,2-b]quinoline- | | |
| 3,14(4H,12H) dione | | |
| hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N[4[[(2amino-5-formyl- | Leucovorin | Seattle, WA |
| 1,4,5,6,7,8 hexahydro4oxo6- | | |
| pteridinyl)methyl]amino]benzoyl], | | |
| calcium salt (1:1)) | | |
| Levamisole HCl | Ergamisol | Janssen Research |
| ((−)-(S)-2,3,5,6-tetrahydro-6- | | Foundation, |
| phenylimidazo [2,1-b] thiazole | | Titusville, NJ |
| monohydrochloride C₁₁H₁₂N₂S•HCl) | | |
| Lomustine | CeeNU | Bristol-Myers |
| (1-(2-chloro-ethyl)-3-cyclohexyl-1- | | Squibb |
| nitrosourea) | | |
| Meclorethamine, nitrogen mustard | Mustargen | Merck |
| (2-chloro-N-(2-chloroethyl)-N- | | |
| methylethanamine hydrochloride) | | |
| Megestrol acetate | Megace | Bristol-Myers |
| 17α(acetyloxy)-6-methylpregna-4,6- | | Squibb |
| diene-3,20-dione | | |
| Melphalan, L-PAM | Alkeran | GlaxoSmithKline |
| (4-[bis(2-chloroethyl) amino]-L- | | |
| phenylalanine) | | |
| Mercaptopurine, 6-MP | Purinethol | GlaxoSmithKline |
| (1,7-dihydro-6H-purine-6-thione | | |
| monohydrate) | | |
| Mesna | Mesnex | Asta Medica |
| (sodium 2-mercaptoethane sulfonate) | | |
| Methotrexate | Methotrexate | Lederle |
| (N-[4-[[(2,4-diamino-6- | | Laboratories |
| pteridinyl)methyl]methylamino]benzoyl]- | | |
| L-glutamic acid) | | |
| Methoxsalen | Uvadex | Therakos, Inc., |
| (9-methoxy-7H-furo[3,2-g][1]- | | Way Exton, Pa |
| benzopyran-7-one) | | |
| Mitomycin C | Mutamycin | Bristol-Myers |
| | | Squibb |
| mitomycin C | Mitozytrex | SuperGen, Inc., |
| | | Dublin, CA |
| Mitotane | Lysodren | Bristol-Myers |
| (1,1-dichloro-2-(o-chlorophenyl)-2-(p- | | Squibb |
| chlorophenyl) ethane) | | |
| Mitoxantrone | Novantrone | Immunex |
| (1,4-dihydroxy-5,8-bis[[2-[(2- | | Corporation |
| hydroxyethyl)amino]ethyl]amino]-9,10- | | |
| anthracenedione dihydrochloride) | | |
| Nandrolone phenpropionate | Durabolin-50 | Organon, Inc., |
| | | West Orange, NJ |
| Nofetumomab | Verluma | Boehringer |
| | | Ingelheim Pharma |
| | | KG, Germany |
| Oprelvekin | Neumega | Genetics Institute, |
| (IL-11) | | Inc., Alexandria, |
| | | VA |
| Oxaliplatin | Eloxatin | Sanofi |
| (cis-[(1R,2R)-1,2-cyclohexanediamine- | | Synthelabo, Inc., |
| N,N′] [oxalato(2-)-O,O′] platinum) | | NY, NY |
| Paclitaxel | TAXOL | Bristol-Myers |
| (5β,20-Epoxy-1,2a,4,7β,10β,13a- | | Squibb |
| hexahydroxytax-11-en-9-one 4,10- | | |
| diacetate 2-benzoate 13-ester with (2R,3S)—N- | | |
| benzoyl-3-phenylisoserine) | | |
| Pamidronate | Aredia | Novartis |
| (phosphonic acid (3-amino-1- | | |
| hydroxypropylidene) bis-, disodium salt, | | |
| pentahydrate, (APD)) | | |
| Pegademase | Adagen | Enzon |
| ((monomethoxypolyethylene glycol | (Pegademase | Pharmaceuticals, |
| succinimidyl) 11-17-adenosine | Bovine) | Inc., Bridgewater, |
| deaminase) | | NJ |
| Pegaspargase | Oncaspar | Enzon |
| (monomethoxypolyethylene glycol | | |
| succinimidyl L-asparaginase) | | |
| Pegfilgrastim | Neulasta | Amgen, Inc |
| (covalent conjugate of recombinant | | |
| methionyl human G-CSF (Filgrastim) and | | |
| monomethoxypolyethylene glycol) | | |
| Pentostatin | Nipent | Parke-Davis |
| | | Pharmaceutical |
| | | Co., Rockville, |
| | | MD |
| Pipobroman | Vercyte | Abbott |
| | | Laboratories, |
| | | Abbott Park, IL |
| Plicamycin, Mithramycin | Mithracin | Pfizer, Inc., NY, |
| (antibiotic produced by Streptomyces | | NY |
| plicatus) | | |
| Porfimer sodium | Photofrin | QLT |
| | | Phototherapeutics, |
| | | Inc., Vancouver, |
| | | Canada |
| Procarbazine | Matulane | Sigma Tau |
| (N-isopropyl-μ-(2-methylhydrazino)-p- | | Pharmaceuticals, |
| toluamide monohydrochloride) | | Inc., Gaithersburg, |
| | | MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi- |
| (recombinant peptide) | | Synthelabo, Inc., |
| Rituximab | Rituxan | Genentech, Inc., |
| (recombinant anti-CD20 antibody) | | South San |
| | | Francisco, CA |
| Sargramostim | Prokine | Immunex Corp |
| (recombinant peptide) | | |
| Streptozocin | Zanosar | Pharmacia & |
| (streptozocin 2-deoxy-2- | | Upjohn Company |
| [[(methylnitrosoamino)carbonyl]amino]- | | |
| a(and b)-D-glucopyranose and 220 mg | | |
| citric acid anhydrous) | | |
| Talc | Sclerosol | Bryan, Corp., |
| (Mg₃Si₄O₁₀(OH)₂) | | Woburn, MA |
| Tamoxifen | Nolvadex | AstraZeneca |
| ((Z)2-[4-(1,2-diphenyl-1-butenyl) | | Pharmaceuticals |
| phenoxy]-N,N-dimethylethanamine 2- | | |
| hydroxy-1,2,3-propanetricarboxylate | | |
| (1:1)) | | |
| Temozolomide | Temodar | Schering |
| (3,4-dihydro-3-methyl-4-oxoimidazo[5,1- | | |
| d]-as-tetrazine-8-carboxamide) | | |
| teniposide, VM-26 | Vumon | Bristol-Myers |
| (4′-demethylepipodophyllotoxin 9-[4,6-0- | | Squibb |
| (R)-2-thenylidene-(beta)-D- | | |
| glucopyranoside]) | | |
| Testolactone | Teslac | Bristol-Myers |
| (13-hydroxy-3-oxo-13,17-secoandrosta- | | Squibb |
| 1,4-dien-17-oic acid [dgr]-lactone) | | |
| Thioguanine, 6-TG | Thioguanine | GlaxoSmithKline |
| (2-amino-1,7-dihydro-6H-purine-6- | | |
| thione) | | |
| Thiotepa | Thioplex | Immunex |
| (Aziridine, 1,1′,1″- | | Corporation |
| phosphinothioylidynetris-, or Tris (1- | | |
| aziridinyl) phosphine sulfide) | | |
| Topotecan HCl | Hycamtin | GlaxoSmithKline |
| ((S)-10-[(dimethylamino) methyl]-4-ethyl- | | |
| 4,9-dihydroxy-1H-pyrano[3′,4′:6,7] | | |
| indolizino [1,2-b] quinoline-3,14- | | |
| (4H,12H)-dione monohydrochloride) | | |
| Toremifene | Fareston | Roberts |
| (2-(p-[(Z)-4-chloro-1,2-diphenyl-1- | | Pharmaceutical |
| butenyl]-phenoxy)-N,N- | | Corp., Eatontown, |
| dimethylethylamine citrate (1:1)) | | NJ |
| Tositumomab, I 131 Tositumomab | Bexxar | Corixa Corp., |
| (recombinant murine immunotherapeutic | | Seattle, WA |
| monoclonal IgG₂ₐ lambda anti-CD20 | | |
| antibody (I 131 is a | | |
| radioimmunotherapeutic antibody)) | | |
| Trastuzumab | Herceptin | Genentech, Inc |
| (recombinant monoclonal IgG₁ kappa anti- | | |
| HER2 antibody) | | |
| Tretinoin, ATRA | Vesanoid | Roche |
| (all-trans retinoic acid) | | |
| Uracil Mustard | Uracil | Roberts Labs |
| | Mustard | |
| | Capsules | |
| Valrubicin, N-trifluoroacetyladriamycin- | Valstar | Anthra --> |
| 14-valerate | | Medeva |
| ((2S-cis)-2-[1,2,3,4,6,11-hexahydro- | | |
| 2,5,12-trihydroxy-7 methoxy-6,11-dioxo- | | |
| [[4 2,3,6-trideoxy-3-[(trifluoroacetyl)- | | |
| amino-α-L-lyxo-hexopyranosyl]oxyl]-2- | | |
| naphthacenyl]-2-oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R—(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl- | | |
| phosphonoethyl) phosphonic acid | | |
| monohydrate) | | |

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, 06-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, S-8184, satraplatin, SB-15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” ninth edition, Eds. Hardman et al., 1996.

In one embodiment, the one or more anticancer agents are selected from the group consisting of abraxane, actinomycin D, aldesleukin, alemtuzumab, alitretinoin, allopurinol, altretamine, amifostine, aminoglutethamide, anastrozole, arsenic trioxide, asparaginase, azacitidine, azathioprine, BCG live, bevacizumab, bexarotene, bicalutamide, bleomycin, bortezomib, busulfan, butazolidin, capecitabine, carboplatin, carmustine, celecoxib, chlorambucil, cisplatin, cladribine, clofarabine, cyclophosphamide, cytarabine, dacarbazine, dactinomycin, darbepoetin alfa, daunomycin, daunorubicin, denileukin diftitox, dexamethasone, dexrazoxane, diethylstilbestrol, docetaxel, doxorubicin, dromostanolone propionate, epirubicin, epoetin alfa, estramustine, ethinyl estradiol, erlotinib, etoposide, exemestane, filgrastim, finasteride, floxuridine, fludarabine, fluorouracil, fluoxymesterone, flutamide, fulvestrant, gefitinib, gemcitabine, gemtuzumab, goserelin acetate, hexamethylmelamine, hydroxychloroquine, hydroxyprogesterone caproate, hydroxyurea, ibritumomab, idarubicin, ifosfamide, imatinib, interferon alfa-2a, interferon alfa-2b, interleukin-2, irinotecan, ketoconazole, lapatinib, lenalidomide, letrozole, leucovorin, leuprolide, levamisole HCl, lomustine, mechlorethamine, medroxyprogesterone acetate, megestrol acetate, meloxicam, melphalan, mercaptopurine, mesna, methotrexate, methoxsalen, methylprednisolone, metronidazole, misonidazole, mithramycin, mitomycin, mitotane, mitoxantrone, nandrolone phenpropionate, nitrogen mustard, nitroimidazole, nitrosourea, nofetumomab, oblimersen sodium, oprelvekin, oxaliplatin, oxaliplatin, oxyphenbutazone, paclitaxel, pamidronate, pazopanib, pegademase, pegaspargase, pegfilgrastim, pentostatin, phenylbutazone, picoplatin, pipobroman, plicamycin, plicamycin, porfimer sodium, prednisolone, prednisone, procarbazine, procarbazine, quinacrine, raloxifene, rasburicase, rituximab, romidepsin, sargramostim, semustine, sorafenib, streptozocin, sunitinib, talc, tamoxifen, temozolomide, teniposide, testolactone, testosterone propionate, thalidomide, thioguanine, thiotepa, tiripazamine, topotecan HCl, toremifene, tositumomab, trastuzumab, tretinoin, trimethoprim/sulfamethoxazole, uracil mustard, valrubicin, vinblastine, vincristine, vindesine, vinorelbine and zoledronic acid.

In another embodiment, the one or more anticancer agents are a taxane. In another embodiment, the taxane is selected from the group consisting of docetaxel and paclitaxel. In another embodiment, the taxane is docetaxel. In another embodiment, the anticancer agent is selected from the group consisting of erlotinib, lenalidomide, cisplatin, erbitux, and oxaliplatin.

In another embodiment, the one or more anticancer agents are selected from the group consisting of topotecan HCl, etoposide, rituximab, carboplatin, 5-fluorouracil, and radiation.

The present invention provides methods for pulsatile dose administration of gossypol with radiation therapy. The term “radiotherapeutic agent,” as used herein refers any type of radiation therapy known to those of skill in the art to be effective for the treatment or amelioration of cancer and/or as an inducer of apoptosis. The invention is not limited by the types, amounts, or delivery and administration systems used to deliver the therapeutic dose of radiation to the patient. For example, the patient may receive photon radiotherapy, particle beam radiation therapy, radioisotope therapy (e.g., radioconjugates with monoclonal antibodies), other types of radiotherapies, and combinations thereof. In some embodiments, the radiation is delivered to the patient using a linear accelerator. In still other embodiments, the radiation is delivered using a gamma knife.

The source of radiation can be external or internal to the patient. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by patients. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The patient may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to a patient, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. The dose of radiation is preferably fractionated for maximal target cell exposure and reduced toxicity.

The total dose of radiation administered to an animal preferably is about 0.01 Gray (Gy) to about 100 Gy. More preferably, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), preferably 1-2 Gy (e.g., 1.5-2 Gy). The daily dose of radiation should be sufficient to induce destruction of the targeted cells. If stretched over a period, radiation preferably is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. Preferably, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present invention.

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In some embodiments of the present invention, gossypol and one or more therapeutic agents are administered to an animal under one or more of the following conditions: at different periodicities, at different durations, at different concentrations, by different administration routes, in separate pharmaceutical compositions, in a single pharmaceutical composition, etc. provided that gossypol is administered according to a discontinuous dosing regimen (i.e., via pulsatile dosing). In one embodiment, the therapeutic agent is an anticancer agent. In some embodiments, gossypol is administered by pulsatile dosing prior to the therapeutic agent, e.g., 0.5, 1, 2, 3, 4, 5, 10, 12, or 18 hours, 1, 2, 3, 4, 5, or 6 days, 1, 2, 3, or 4 weeks prior to the administration of the therapeutic agent. In some embodiments, gossypol is administered by pulsatile dosing after the therapeutic agent, e.g., 0.5, 1, 2 3, 4, 5, 10, 12, or 18 hours, 1, 2, 3, 4, 5, or 6 days, 1, 2, 3, or 4 weeks after the administration of the therapeutic agent. In some embodiments, gossypol and the therapeutic agent are administered concurrently but on different schedules, e.g., gossypol is administered on at least two consecutive days followed by at least one day wherein gossypol is not administered while the therapeutic agent is administered once a week, once every two weeks, once every three weeks, or once every four weeks, and as separate pharmaceutical compositions. In other embodiments, gossypol is administered on one day a week while the therapeutic agent is administered daily, once a week, once every two weeks, once every three weeks, or once every four weeks. In one embodiment, (−)-gossypol is administered twice-a-day for three consecutive days every 21 days, one therapeutic agent, e.g., docetaxel, is administered every 21 days, and another therapeutic agent, e.g., prednisone, is administered daily. In a particular embodiment, (−)-gossypol is administered on day 1, day 2, and day 3 of a treatment cycle and an anticancer agent is administered on day 1 of the treatment cycle. The treatment cycle may be repeated one or more times.

The above-described dose administration schedules are provided for illustrative purposes only and should not be considered limiting.

Pharmaceutical compositions may be produced by combining gossypol in a therapeutically effective amount to induce apoptosis in cells or to sensitize cells to inducers of apoptosis with a pharmaceutically acceptable carrier. Pharmaceutical compositions may comprise, for example, (±)-gossypol, (+)-gossypol, (−)-gossypol, (±)-gossypol co-crystal, (+)-gossypol co-crystal or (−)-gossypol co-crystal. Pharmaceutical compositions may also comprise one or more additional therapeutic agents.

Pharmaceutical compositions useful within the scope of this invention include all compositions wherein gossypol is contained in an amount which is effective to achieve its intended purpose, e.g., to treat, ameliorate or prevent a disease, condition or disorder responsive to the induction of apoptosis. While individual needs vary, determination of optimal ranges of effective amounts of each component in a pharmaceutical composition is within the skill of the art. In one embodiment, the pharmaceutical composition comprising gossypol may be administered to patients, e.g. humans, orally at a dose totaling about 1 mg to about 1200 mg, or an equivalent dose of the pharmaceutically acceptable salt thereof, per day. In another embodiment, a total oral dose of about 5 mg to about 500 mg, about 5 mg to about 250 mg, about 5 mg to about 100 mg, or about 5 mg to about 60 mg is administered per day. In another embodiment, a total oral dose of about 90 mg to about 240 mg is administered per day. In another embodiment, a total oral dose of about 80 mg to about 200 mg is administered per day. In another embodiment, an oral dose of about 5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg or 200 mg is administered two times a day. In another embodiment, an oral dose of 20 mg, 30 mg, 35 mg, 40 mg, 45 mg, 50 mg, 55 mg, 60 mg, 65 mg, 70 mg, 75 mg or 80 mg is administered two times a day. In another embodiment, an oral dose of about 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg is administered once a day. In another embodiment, an oral dose of 80 mg, 90 mg, 100 mg, 110 mg, 120 mg, 130 mg, 140 mg, 150 mg, 160 mg, 170 mg, 180 mg, 190 mg or 200 mg is administered once a day. In certain embodiments, the amount (e.g., dose) of gossypol administered may increase as the period of time between dosing increases, since the potential for adverse events may decrease under such circumstances.

The unit oral dose may comprise from about 1 mg to about 1000 mg of the pharmaceutical composition comprising gossypol. In one embodiment, the unit oral dose may comprise from about 5 mg to about 500 mg of the pharmaceutical composition comprising gossypol. In another embodiment, the unit oral dose may comprise from about 5 mg to about 100 mg. In another embodiment, the unit oral dose may comprise from about 5 mg to about 30 mg. In another embodiment, the unit oral dose may comprise from about 30 mg to about 80 mg. In another embodiment, the unit oral dose may comprise about 40 mg to about 60 mg. The unit dose may be administered one or more times daily as one or more tablets, capsules and the like, each containing from about 1 to about 1000 mg of the pharmaceutical composition comprising gossypol, conveniently about 5 mg to about 100 mg of the composition, e.g., about 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg. In one embodiment, the unit oral dose is administered two times per day. In another embodiment, the unit oral dose is administered two times per day for three consecutive days. In another embodiment, the unit oral dose is administered for five consecutive days.

For intramuscular injection, the dose is generally about one-half of the oral dose. For example, a suitable intramuscular dose of gossypol would be about 0.5 mg to about 500 mg. In one embodiment, the intramuscular dose would be about 0.5 mg to about 100 mg, about 0.5 to about 50 mg, about 0.5 mg to about 25 mg, or about 0.5 mg to about 15 mg.

In a topical formulation, the composition may be present at a concentration of about 0.01 to 100 mg per gram of carrier. In one embodiment, the composition is present at a concentration of about 0.07 mg/g to about 1.0 mg/g, about 0.1 to about 0.5 mg/g, or about 0.4 mg/g.

Gossypol may be administered via pulsatile dosing as part of a pharmaceutical composition containing suitable pharmaceutically acceptable carriers comprising excipients and auxiliaries which facilitate processing of the compositions into preparations which can be used pharmaceutically. Pharmaceutical preparations which can be administered orally include tablets, dragees, slow release lozenges, capsules and the like. Pharmaceutical preparations which can be administered topically include mouth rinses and mouth washes, gels, liquid suspensions, hair rinses, hair gels, shampoos and the like. Pharmaceutical preparations which can be administered rectally include suppositories and the like. Pharmaceutical preparations for administration by injection include suitable solutions and the like. Pharmaceutical preparations administered via pulsatile dosing contain from about 0.01 to about 99 percent, or from about 0.25 to about 75 percent of gossypol, together with the excipient(s).

Methods of the invention may be administered to any subject or patient which may experience the beneficial effects of such methods. Foremost among such patients are mammals, e.g., humans, although the invention is not intended to be so limited. Other patients include veterinary animals (cows, sheep, pigs, horses, dogs, cats and the like).

Gossypol and pharmaceutical compositions thereof may be administered via pulsatile dose administration by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired. In a particular embodiment, gossypol is orally administered.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams are preferably formulated from a mixture of mineral oil, self-emulsifying beeswax and water in which mixture the active ingredient, dissolved in a small amount of an oil such as almond oil, is admixed. A typical example of such a cream is one which includes about 40 parts water, about 20 parts beeswax, about 40 parts mineral oil and about 1 part almond oil.

Ointments may be formulated by mixing a suspension of the active ingredient in a vegetable oil such as almond oil with warm soft paraffin and allowing the mixture to cool. A typical example of such an ointment is one which includes about 30% almond oil and about 70% white soft paraffin by weight.

Lotions may be conveniently prepared by preparing a suspension of the active ingredient in a suitable high molecular weight alcohol such as propylene glycol or polyethylene glycol.

The following examples are illustrative, but not limiting, of the methods of the present invention. Suitable modifications and adaptations of the variety of conditions and parameters normally encountered in clinical therapy and which are obvious to those skilled in the art are within the spirit and scope of the invention.

### Example 1

### Pulsatile Dosing of Gossypol

A phase I clinical trial was carried out to compare the maximum tolerated dose and safety of daily (i.e., continuous) versus pulsatile (i.e., intermittent) dosing of (−)-gossypol in patients with advanced cancer. A secondary objective of this study was to identify any anti-tumor activity of (−)-gossypol. Patients were treated with increasing doses of (−)-gossypol according to the following dosing schedules: “Daily” dosing: 5 to 60 mg/day of (−)-gossypol on 21 days per 28 day cycle; “BID×3d” dosing: 30 to 80 mg BID of (−)-gossypol on 3 consecutive days (e.g., Monday-Tuesday-Wednesday) repeated every other week per 28 day cycle; and “Weekly” dosing: 80 to 200 mg of (−)-gossypol once weekly per 28 day cycle. Adverse events (AEs) were graded by NCI-CTCAE v3. Overall, pulsatile dosing (BID×3d and Weekly) resulted in a reduced percentage of AEs, particularly Grade 3/4 AEs, as compared to continuous daily dosing (see Table 2, Any AE).

TABLE 2
(−)-Gossypol Dose Schedule

| | Daily | | BID × 3 d | | Weekly | |
|---|---|---|---|---|---|---|
| | N = 38 | | N = 21 | | N = 12 | |
| | Grade | | Grade | | Grade | |
| Adverse Event | ½ | ¾ | ½ | ¾ | ½ | ¾ |
| (AE) | N (%) | N (%) | N (%) | N (%) | N (%) | N (%) |
| Any AE | 38 (100) | 37 (98) | 16 (76) | 11 (52) | 10 (83) | 10 (48) |
| Nausea | 22 (58) | 5 (13) | 14 (67) | 0 | 2 (17) | 0 |
| Vomiting | 13 (34) | 7 (18) | 9 (43) | 2 (10) | 0 | 0 |
| Diarrhea | 13 (34) | 5 (13) | 8 (38) | 1 (5) | 0 | 0 |
| Abdominal pain | 8 (21) | 1 (3) | 5 (24) | 2 (10) | 2 (17) | 0 |
| Constipation | 9 (24) | 0 | 6 (29) | 0 | 4 (33) | 0 |
| Abdominal distention | 4 (11) | 0 | 3 (14) | 0 | 3 (25) | 0 |
| Small intestinal | 0 | 4 (11) | 0 | 1 (5) | 0 | 2 (8) |
| obstruction (ileus) | | | | | | |
| Abdominal discomfort | 0 | 2 (5) | 1 (5) | 0 | 0 | 0 |
| Pancreatitis | 0 | 0 | 0 | 1 (5) | 0 | 0 |
| Dry mouth | 3 (8) | 0 | 2 (10) | 0 | 1 (8) | 0 |
| Flatulence | 1 (3) | 0 | 2 (10) | 0 | 1 (8) | 0 |
| Fatigue/Asthenia | 31 (81) | 4 (11) | 15 (72) | 1 (5) | 8 (67) | 1 (8) |
| Pain | 6 (16) | 0 | 3 (14) | 1 (5) | 0 | 0 |
| Pyrexia | 4 (11) | 1 (3) | 3 (14) | 0 | 0 | 1 (8) |
| Peripheral edema | 5 (13) | 0 | 0 | 0 | 3 (25) | 0 |
| Pneumatosis | 0 | 0 | 0 | 1 (5) | 0 | 1 (8) |
| intestinalis | | | | | | |
| Anorexia or | 19 (50) | 1 (3) | 10 (48) | 1 (5) | 7 (58) | 0 |
| decreased appetite | | | | | | |
| Dehydration | 9 (24) | 2 (5) | 3 (14) | 2 (10) | 2 (17) | 2 (17) |
| Hypokalemia | 1 (3) | 3 (8) | 1 (5) | 3 (14) | 2 (17) | 1 (8) |
| Hyperkalemia | 2 (5) | 0 | 1 (5) | 1 (5) | 0 | 0 |
| Hypocalcemia | 2 (5) | 2 (5) | 0 | 0 | 2 (17) | 0 |
| Hyponatremia | 1 (3) | 1 (3) | 1 (5) | 1 (5) | 0 | 0 |
| AST increase | 6 (16) | 6 (13) | 2 (10) | 0 | 1 (8) | 4 (33) |
| Creatinine | 0 | 0 | 0 | 1 (5) | 0 | 1 (8) |
| phosphokinase | | | | | | |
| increase | | | | | | |
| Creatinine increased | 6 (16) | 0 | 2 (10) | 0 | 2 (17) | 1 (8) |
| Alkaline | 6 (16) | 0 | 2 (10) | 0 | 2 (17) | 1 (8) |
| phosphatase | | | | | | |
| increase | | | | | | |
| ALT increase | 7 (18) | 5 (13) | 2 (10) | 0 | 3 (25) | 1 (8) |
| Troponin I or T | 2 (5) | 1 (3) | 1 (5) | 0 | 1 (8) | 2 (17) |
| increased | | | | | | |
| Albumin decreased | 0 | 0 | 0 | 0 | 0 | 1 (8) |
| Weight decreased | 11 (29) | 0 | 4 (19) | 0 | 2 (17) | 0 |
| White blood cells | 0 | 0 | 2 (10) | 0 | 0 | 0 |
| increased | | | | | | |
| Dyspnea | 6 (16) | 3 (8) | 5 (24) | 0 | 1 (8) | 1 (8) |
| Pleural Effusion | 1 (3) | 2 (5) | 0 | 0 | 0 | 1 (8) |
| Cough | 8 (21) | 0 | 0 | 0 | 0 | 0 |
| Infection | 1 (3) | 1 (3) | 0 | 0 | 0 | 1 (8) |
| Pneumonia | 0 | 1 (3) | 0 | 1 (5) | 0 | 0 |
| Urinary Tract | 5 (13) | 0 | 2 (10) | 0 | 2 (17) | 0 |
| Infection | | | | | | |
| Sinusitis | 4 (11) | 0 | 0 | 0 | 0 | 0 |
| Hyperbilirubinaemia | 2 (5) | 1 (3) | 0 | 1 (5) | 2 (17) | 2 (17) |
| Renal Failure | 0 | 0 | 0 | 1 (5) | 1 (8) | 0 |
| Proteinurea | 0 | 0 | 2 (10) | 0 | 2 (17) | 0 |
| Hepatic | 0 | 0 | 0 | 1 (5) | 0 | 1 (8) |
| encephalopathy | | | | | | |
| Dizziness | 7 (18) | 0 | 2 (10) | 0 | 2 (17) | 0 |
| Headache | 5 (13) | 0 | 1 (5) | 0 | 1 (8) | 0 |
| Dysgeusia | 2 (5) | 0 | 2 (10) | 0 | 2 (17) | 0 |
| Back pain | 6 (16) | 0 | 1 (5) | 0 | 1 (8) | 0 |
| Insomnia | 7 (18) | 0 | 1 (5) | 0 | 3 (25) | 0 |
| Mental status | 0 | 1 (3) | 0 | 0 | 0 | 1 (8) |
| changes | | | | | | |

### Example 2

### Clinical Efficacy of Gossypol

Following (−)-gossypol administration to patients with advanced cancer, clinical efficacy (e.g., patients having stable disease for 60 days or more) was monitored according to the following dosing schedules: “Daily” dosing: 5 to 60 mg/day of (−)-gossypol on 21 days per 28 day cycle; “BID×3d” dosing: 30 to 80 mg BID of (−)-gossypol on 3 consecutive days (e.g., Monday-Tuesday-Wednesday) repeated every other week per 28 day cycle; and “Weekly” dosing: 80 to 200 mg of (−)-gossypol once weekly per 28 day cycle. Pulsatile dosing (BID×3d) resulted in a longer median duration of days of stable disease as compared to continuous daily dosing (Table 3). Tumor types represented in this study included: non-small cell lung cancer, non-Hodgkin lymphoma, chronic lymphocytic leukemia/small lymphocytic lymphoma, colon cancer, breast cancer, small cell lung cancer, head and neck cancer, sarcoma, hepatocellular cancer, pancreatic cancer, esophageal cancer, cholangiocarcinoma, carcinoid/neuroendocrine cancer, Hodgkin's lymphoma, endometrial cancer, adrenal cancer, melanoma, gastrointestinal stromal tumor, renal cancer, bladder cancer, and ovarian cancer.

TABLE 3
(−)-Gossypol Clinical Efficacy

| | Daily | BID × 3 d | Weekly |
|---|---|---|---|
| | N = 38 | N = 21 | N = 12 |
| Median # (%) of | 6 (16) | 6 (24) | 2 (13) |
| patients with stable | | | |
| disease ≧60 days | | | |
| Median duration of | 82 (56-341) | 180 (72-443) | 69 (58-80) |
| days of stable | | | |
| disease (range) | | | |

### Example 3

### In Vivo Efficacy of (−)-Gossypol Acetic Acid Co-Crystals in the A549 Non-Small Cell Cancer (NSCLC) Xenograft Model

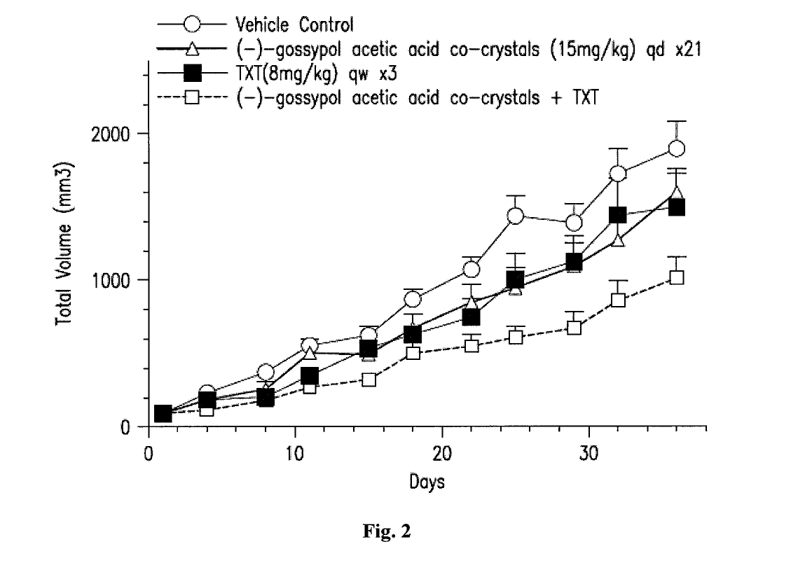

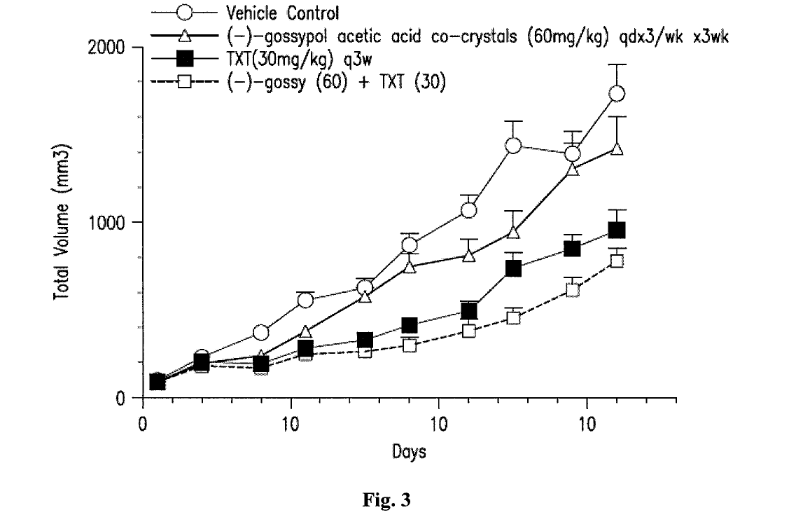

The in vivo efficacy of (−)-gossypol acetic acid co-crystals alone or in combination with taxotere (TXT) in the A549 NSCLC xenograph model is shown in FIGS. 2 and 3. About 5 million cells of A549 were inoculated into nude mice, 8 mice per dosing group. In one experiment, (−)-gossypol acetic acid co-crystals were administered at 15 mg/kg, oral dosing (po), daily for 21 days, either alone or in combination with taxotere at 8 mg/kg, iv, once a week for three weeks (FIG. 2). In another experiment, (−)-gossypol acetic acid co-crystals were administered at 60 mg/kg, po, daily for three days per week (day 1-3/week) every two weeks (days 1-3, and then days 15-17), either alone or in combination with taxotere at 30 mg/kg, iv, single dose only, once every three weeks (FIG. 3). The results of these studies show inter alia that an intermittent dosing of (−)-gossypol acetic acid co-crystals in combination with taxotere effectively reduces tumor volume.

### Example 4

### Clinical Study of Gossypol in Combination with Docetaxel

An open-label non-randomized Phase I-II clinical trial (labeled CS-202) is being carried out on patients with histologically confirmed metastatic prostate cancer. Briefly, patients are divided in into two cohorts: Cohort A-Patients with rising PSA level on hormonal therapy, i.e., hormone-refractory patients, and Cohort B-Patients with PSA or disease progression on docetaxel (taxotere), i.e., docetaxel-refractory patients. All patients were treated with 40-60 mg (−)-gossypol acetic acid co-crystal 40-60 mg BID days 1-3, every 21 days, 75 mg/m2 docetaxel q 21 days and prednisone, daily per label.

Phase I data from 9 patients is summarized as follows: no dose-limiting toxicities, no severe gastrointestinal toxicity, no increase in docetaxel hematological toxicity.

Phase I-II data is available on first 20 patients of Cohort A. The patient demographics are provided in Table 4. See N. Engl. J. Med. 351:1502-1512 (2004) for further details of TAX 327 clinical study.

TABLE 4

| | | CS-202 CoA | TAX 327 |
|---|---|---|---|
| Characteristic | | N = 20 | N = 335 |
| Age | Median | 69.5 | 68 |
| | Range | 55-84 | 42-92 |
| | >75, % | 40 | 20 |
| Gleason Score, % | <=7 | 30 | 42 |
| | 8, 9, or 10 | 40 | 31 |
| | NA | 30 | 26 |
| Hormonal | 1 | 15 | 9 |
| Therapy, % | 2 | 55 | 68 |
| | >2 | 30 | 23 |
| PSA | Median | 174 | 114 |
| | >=20 ng/ml, % | 100 | 87 |
| KPS/ECOG, % | | ECOG 2: 0 | KPS <= 70: 13 |
| Pain, % | PPI score >=2 | N/A | 45 |
| | Present | N/A | N/A |
| Extent | Bone Mets | 75 | 90 |
| of Disease, % | Visceral Disease | 65 | 22 |
| Evidence | PSA Increase | 100 | 72 |
| of PD at entry, % | | | |

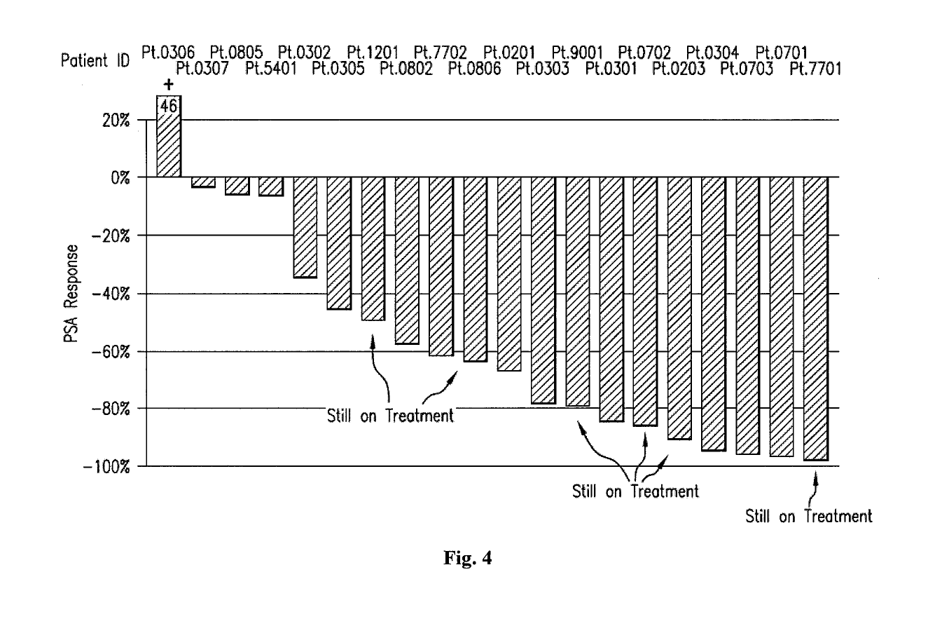

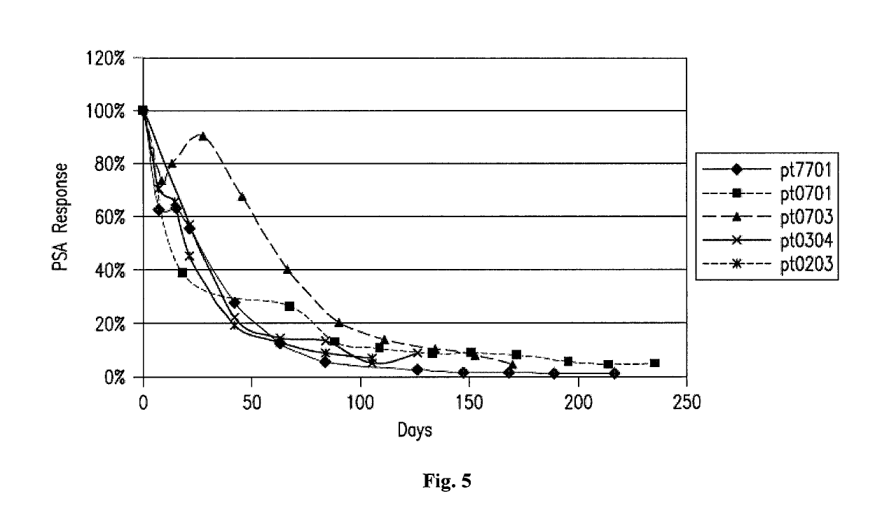

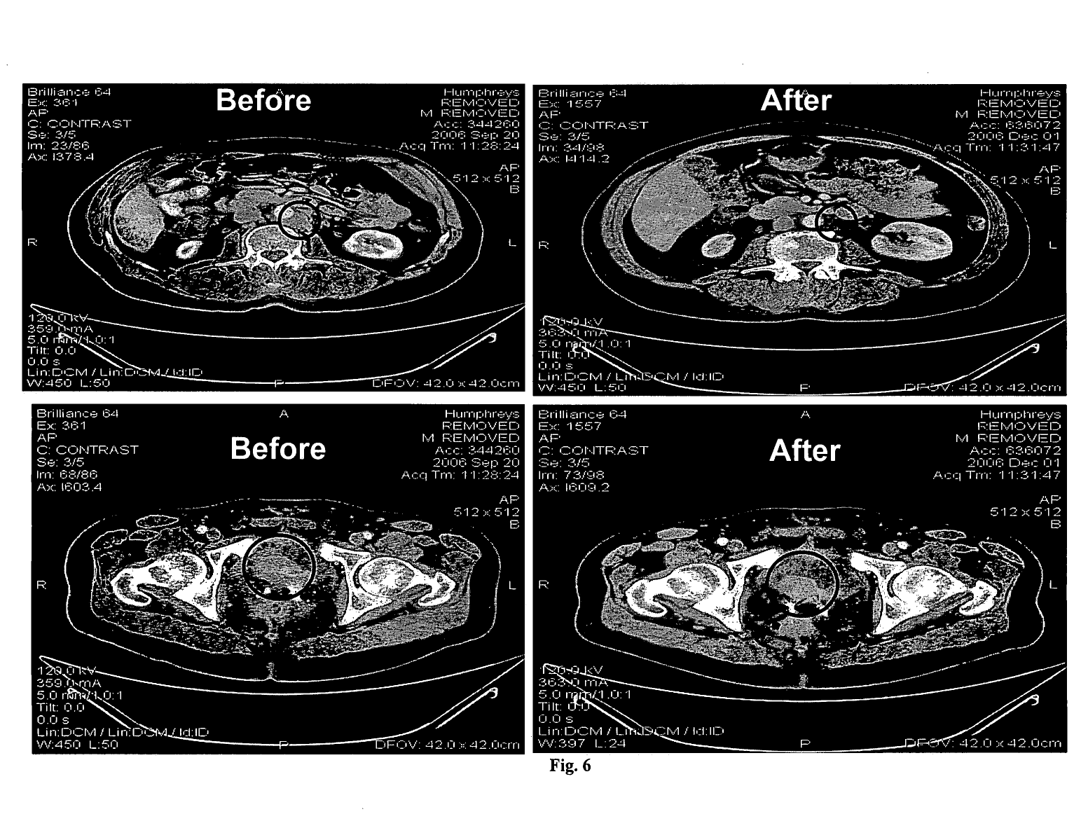

FIG. 4 is a waterfall plot illustrating the therapeutic response of the first 20 patients of Cohort A. These data indicate a high rate of PSA response with (−)-gossypol acetic acid co-crystal treatment. In addition, only one patient out of 20 was refractory at the onset of treatment. Data from this study also indicates steep declines in the slope of PSA response versus time and PSA reductions occur early in the treatment and are long lasting (FIG. 5). Finally, CT scanning shows tumor size reduction following treatment with (−)-gossypol, docetaxel, and prednisone (FIG. 6).

Table 5 shows the preliminary efficacy and safety comparisons between CS-202 and TAX 327. Patients receiving (−)-gossypol acetic acid co-crystal (labeled “(−)-gossy”) show a marked improvement in 50% PSA reduction and a significantly lower refractory rate. (“D”=docetaxel; “P”=prednisone).

TABLE 5

| | CS-202 | TAX 327 |
|---|---|---|
| | N = 20/36 | N = 335 |
| | D/P + (−)-gossy | D/P |
| Median Baseline PSA, ng/mL | 174 | 114 |
| 50% PSA Reduction, n/N (%) or % | 14/20 (70) | 45 |
| 95% CI, % | 50-90 | 40-50 |
| 30% PSA Reduction, n/N (%) or % | 16/20 (80) | 67 |
| PSA Normalization, n/N (%) or % | 3+/20 (15+) | 14 |
| PSA Refractory, n/N (%) or % | 1/20 (5) | 22 |
| Measurable Disease, n/N (%) or % | 3/7 (43) | 12 |
| PR | | |
| Median PFS, mo | N/A | N/A |
| Median OS, mo | N/A | 18.9 |
| Pts on Rx = 10 Cycles, n/N (%) or % | 6+/20 (30+) | 46 |
| Pts disc. for PD, n/N (%) or % | 7+/20 (35+) | 38 |
| Pts disc. for AE, n/N (%) or % | 4+/20 (20+) | 11 |

Table 6 compares the PSA responses of (−)-gossypol acetic acid co-crystal Cohort A combination therapy with approved docetaxel monotherapy regimens and other investigational combination therapies for the treatment of hormone refractory prostate cancer. (−)-Gossypol acetic acid co-crystal (labeled “(−)-gossypol”) responses compare favorably to those reported in these other studies.

TABLE 6

| | Taxotere + | Taxotere + | (−)-gossypol + | DN-101+ | Avastin |
|---|---|---|---|---|---|
| | Prednisone | Estramustine | Taxotere and | Taxotere + | Taxotere + |
| | (Rand Phase 3) | (SWOG 9916) | Prednisone | Prednisone | Estramustine |
| Median Baseline | 120^{1,2} | 84³ | 174 | 121 | N/A |
| PSA (ng/mL) | | | | | |
| Median Days to | 163² | <90⁴ | 42.5 | 87 | N/A |
| PSA Response | | | | | |
| PSA Response | 45%^{1,2} | 50%³ | 70% | 63%² | 81% |
| Rate (50% | @ 3 mos | @ 3 mos | @ 3 mos | | |
| Decline) | | | | | |
| Pts with 30% | 67% | 76% | 80% | N/A | N/A |
| PSA Decline | @ 3 mos⁵ | @ 3 mos⁴ | @ 3 mos | | |
| Pts with 90% | 13%⁵ | 15%⁴ | 25% | N/A | N/A |
| PSA Decline | | | | | |
| RECIST PR % | 12%^{1,2} | 17%³ | ≧43% | 29% | N/A |

¹N Engl J Med 351: 1502-1512, 2004;
²J Clin Oncol 25: 669-674, 2007;
³N Engl J Med 351: 1513-1520, 2004;
⁴J Natl Cancer Inst 98: 516-521, 2006;
⁵J Clin Oncol 25: 3965-3970, 2007

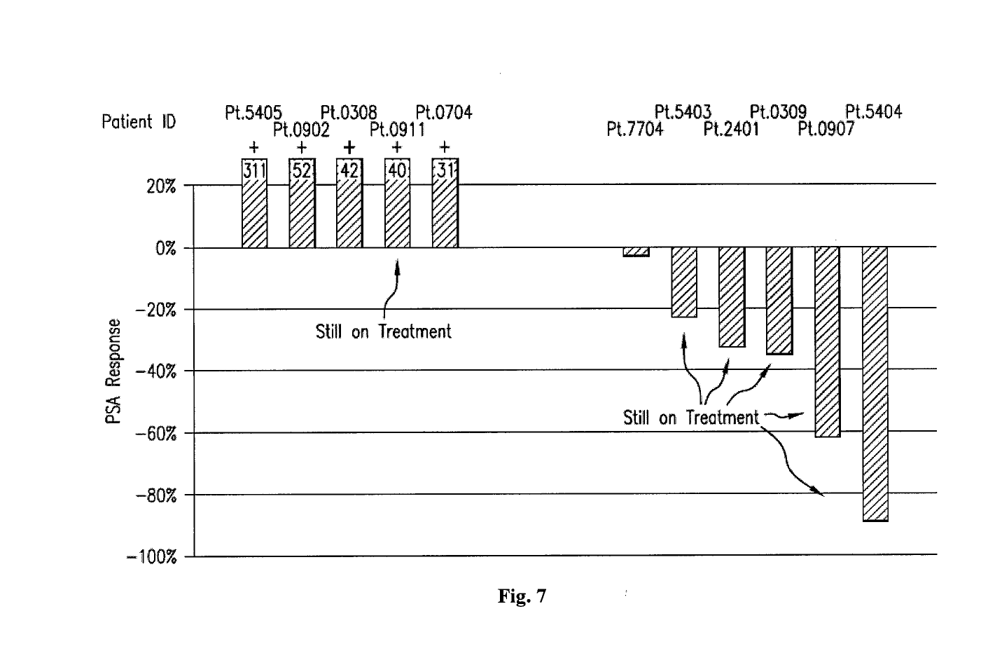

FIG. 7 illustrates the therapeutic response of the first 11 patients of Cohort B. These data indicate the desired PSA response with (−)-gossypol acetic acid co-crystal treatment in 6 out to the 11 patients, with 4 patients having a 30+% decline and 2 patients having a 50+% decline in PSA response.

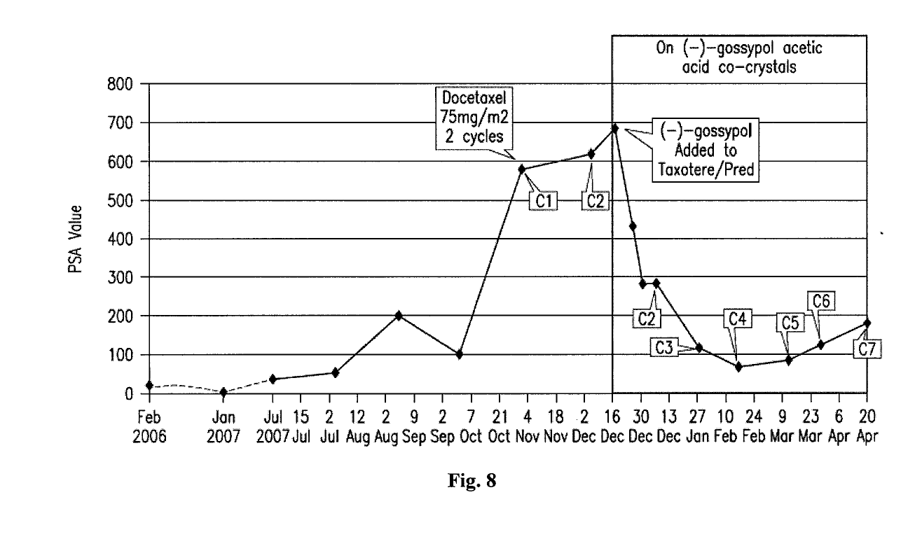

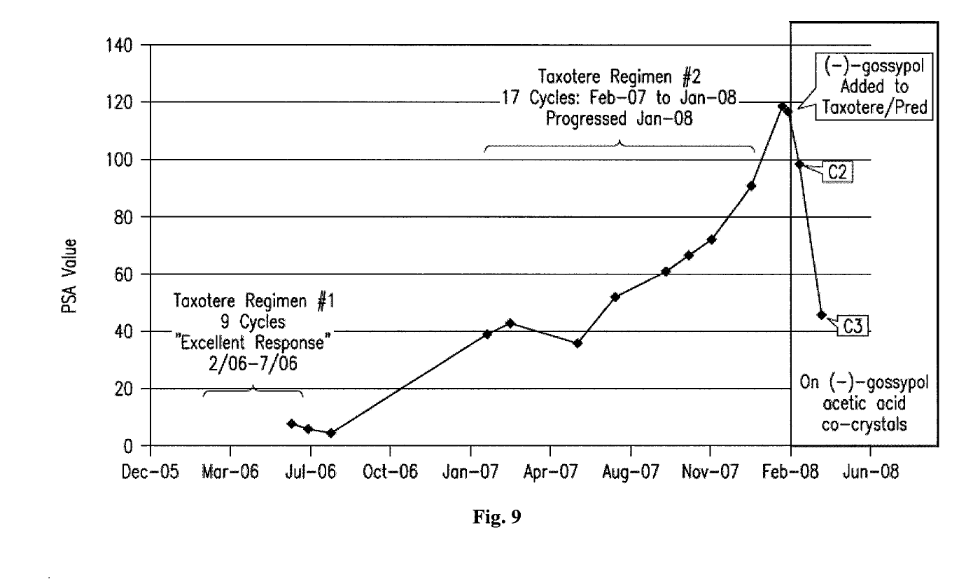

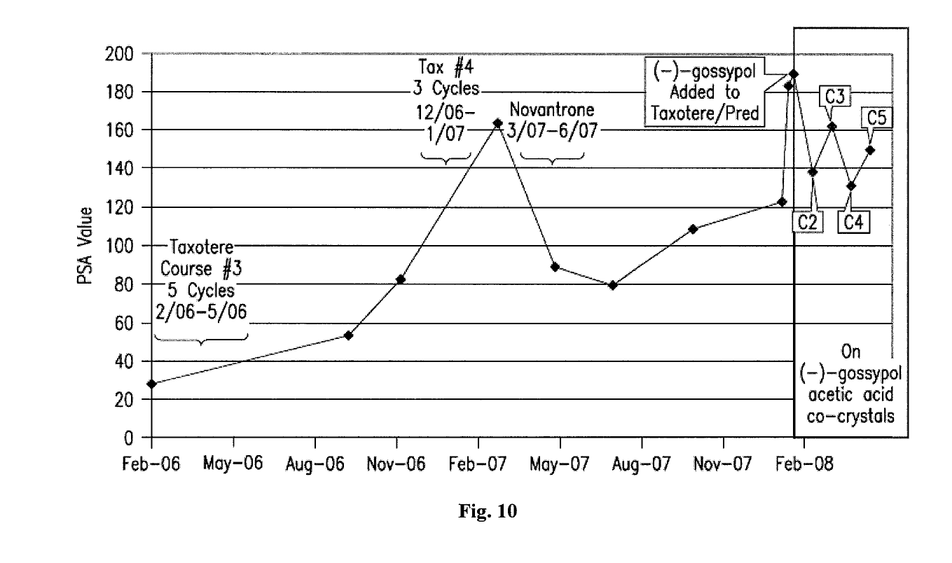

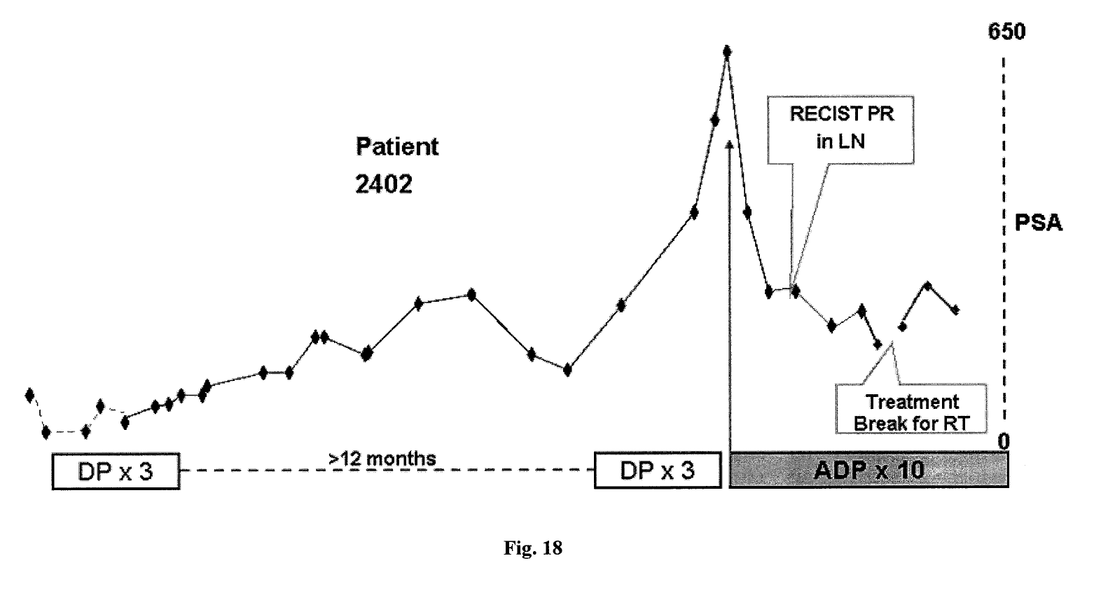

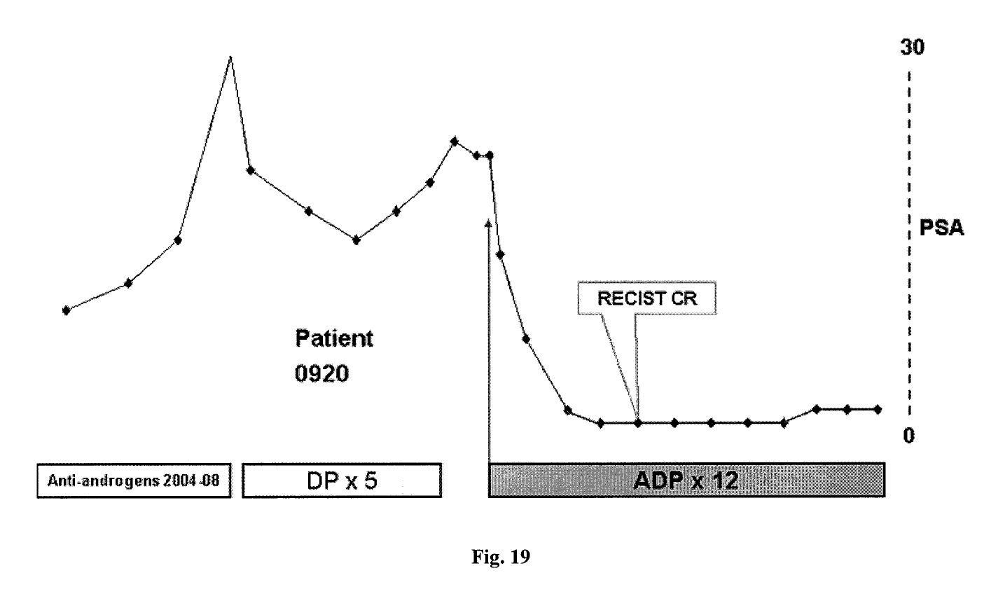

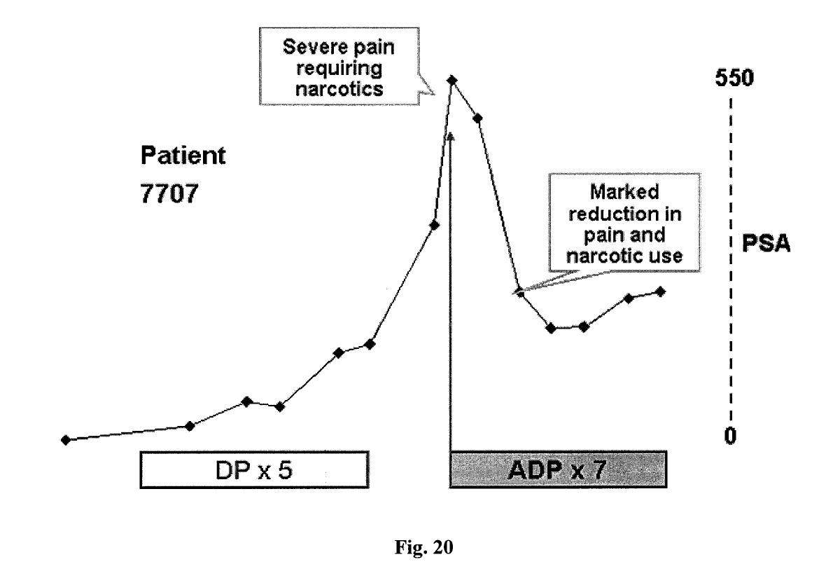

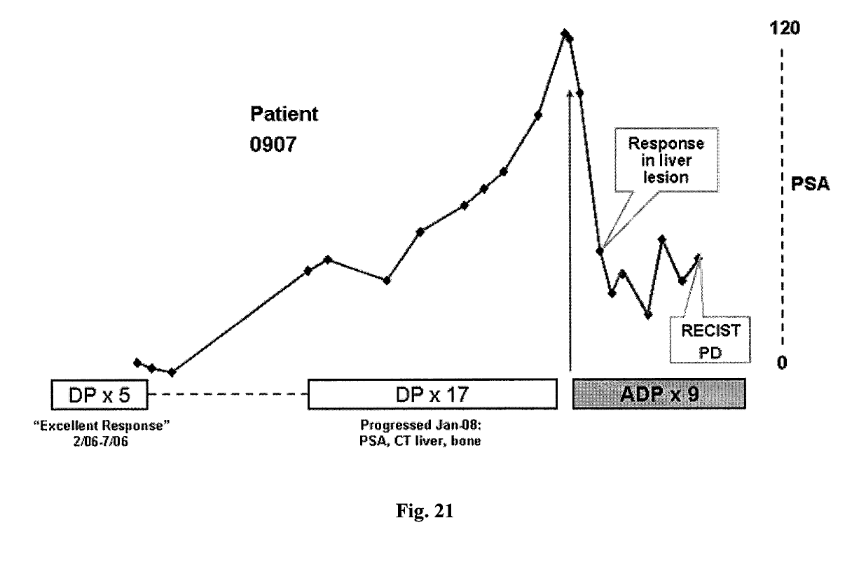

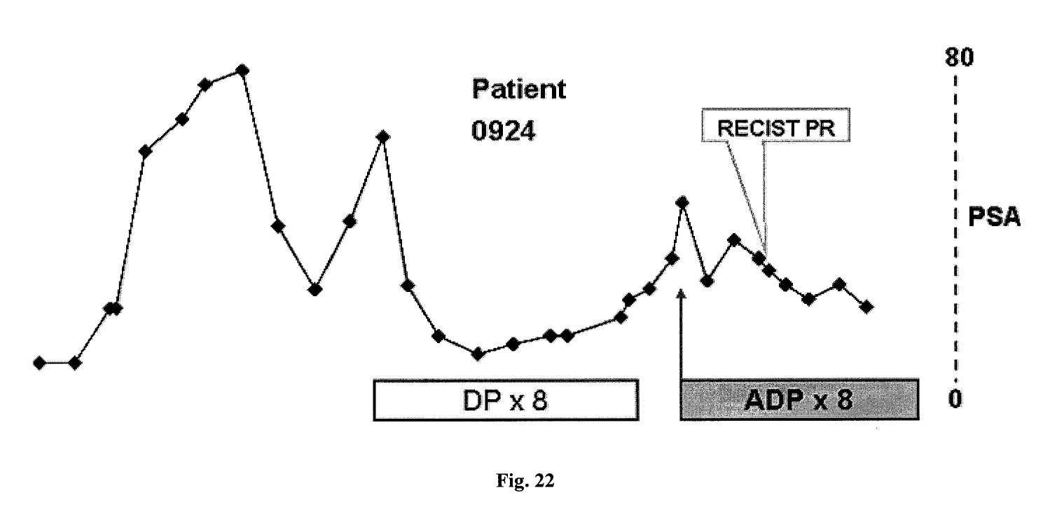

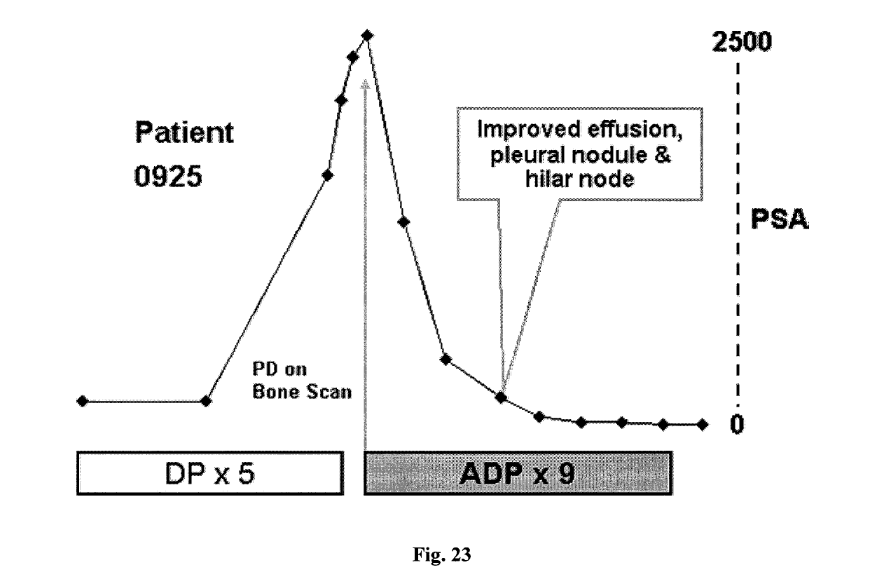

FIGS. 8-10 and 18-23 illustrate the rapid therapeutic response when (−)-gossypol acetic acid co-crystal is added to a taxotere/prednisone regimen of various patients of Cohort B. The abrupt reversal of the PSA response (from rising to falling) suggests pulsatile dosing of (−)-gossypol acetic acid co-crystal may be especially effective in treating chemoresistant patients.

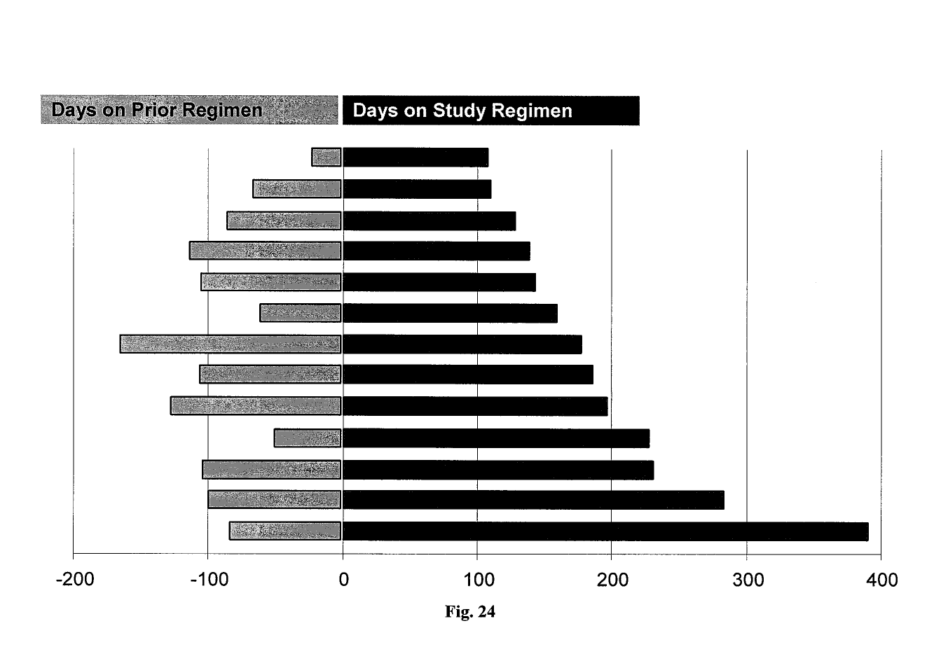

FIG. 24 shows an inversion analysis, noting that many patients (13 out of 37) have been on the AT-101/taxotere/prednisone regimen longer than their prior taxotere/prednisone regimen without AT-101.

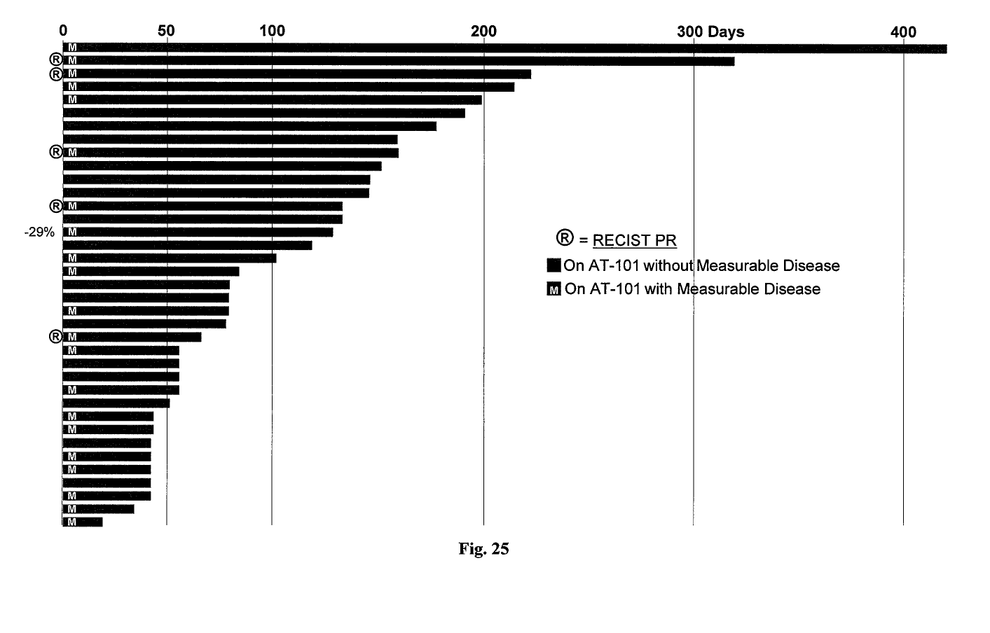

FIG. 25 is a time-to-event plot in taxotere-refractory patients (37 eligible patients; 21 with measurable disease). These data show multiple durable responses in truly refractory patients when AT-101 is added to taxotere/prednisone regimen and RECIST partial responses are observed. Treatment regimen in this study is: oral AT-101 40 mg BID×3 days q 3 weeks, IV taxotere 75 mg/m² q 3 weeks, and oral prednisone 5 mg BID daily.

### Example 5

### Clinical Study of Gossypol for the Treatment of Cancer via Pulsatile Dose Administration

Following pulsatile dose administration of (−)-gossypol to patients with advanced cancer, clinical efficacy (e.g., patients having stable disease for 60 days or more) was monitored. Among 37 patients treated with AT-101 ((−)-gossypol acetic acid co-crystal) b.i.d. (twice daily) times 3 days every other week (EoW), two patients had clinical benefit as determined by a prolonged progression free period of more than 1 year. The case summaries are as follows:

Patient 1 is a 52 year old male Caucasian diagnosed with non-small cell lung cancer (NSCLC) by bronchial biopsy in March 2005. The patient initially received the Carboplatin/paclitaxel with radiation therapy as adjuvant therapy from April to May 2005. Disease progression was diagnosed in September 2005 and treatment with erlotinib was begun in October 2005. The patient had progressive disease in January 2006. The patient started on treatment with AT-101 40 mg b.i.d. times 3 days every other week in March 2006. When the patient started on treatment with AT-101 he had extensive lymph node disease that remained stable on study drug for approximately 939 days (2.6 years). The patient's baseline signs and symptoms included: anemia, fatigue and productive cough. His medical history includes: history of smoking, COPD, BPH, hypercholesterolemia, hypophosphatemia, RT induced esophagitis. The only AT-101 on-treatment toxicities this patient experienced was grade 2 peripheral neuropathy.

Patient 2 is a 64 year old male Caucasian diagnosed with a neuroendocrine carcinoma by a CT guided biopsy of an abdominal mass in May 2002. The patient was initially treated with interferon alpha from September 2002 to March 2003 but the disease progressed. Subsequently, the patient was treated with an investigational VEGFR2 targeted agent, SUO11248, from August 2003 to March 2004 until progression. The patient next received interferon plus capecitabine from August 2004 to June 2005 until disease progression in July 2005. Next, the patient was treated with lysosomal doxirubicin from July 2005 to January 2006 until disease progression in February 2006. He started on the AT-101 treatment in February 2006 and received AT-101 30 mg b.i.d. times 3 days every other week. At the start of therapy, disease was present in the liver, abdomen, and pelvis and notable for numerous mesenteric masses. These remained stable on study for 18 cycles (534 days). The patient came off study due to increased weight loss and the investigator decision to send the patient for surgery for tumor debulking. The patient's baseline signs and symptoms included tumor pain, fatigue and weight loss. The patient's medical history included: GERD, HTN, congenital pulmonary cysts, anemia, and diarrhea. On treatment toxicities included the following grade 1 toxicities: general body aches, nausea, flatulence, headache, fatigue, hypomagnesemia, vomiting, hypokalemia and diarrhea. The patient also experienced the following grade 2 toxicities: weight loss, nausea, vomiting and grade 3 small bowel obstruction considered serious. The small bowel obstruction occurred just prior to coming off study for tumor debulking, and was determined to be unrelated to AT-101 treatment.

### Example 6

### Clinical Study of Gossypol in Combination with Docetaxel for the Treatment of Non-Small Cell Lung Cancer

A randomized Phase II, two-arm, double-blind study of AT-101 in combination with docetaxel versus docetaxel plus placebo in patients with relapsed or refractory non-small cell lung cancer is underway. A preliminary analysis performed on the study's primary endpoint, progression free survival (PFS), based on available data in the clinical database is presented. The disease status and date of progressive disease, if detected, were as reported by the participating investigators. Available overall survival data were also analyzed.

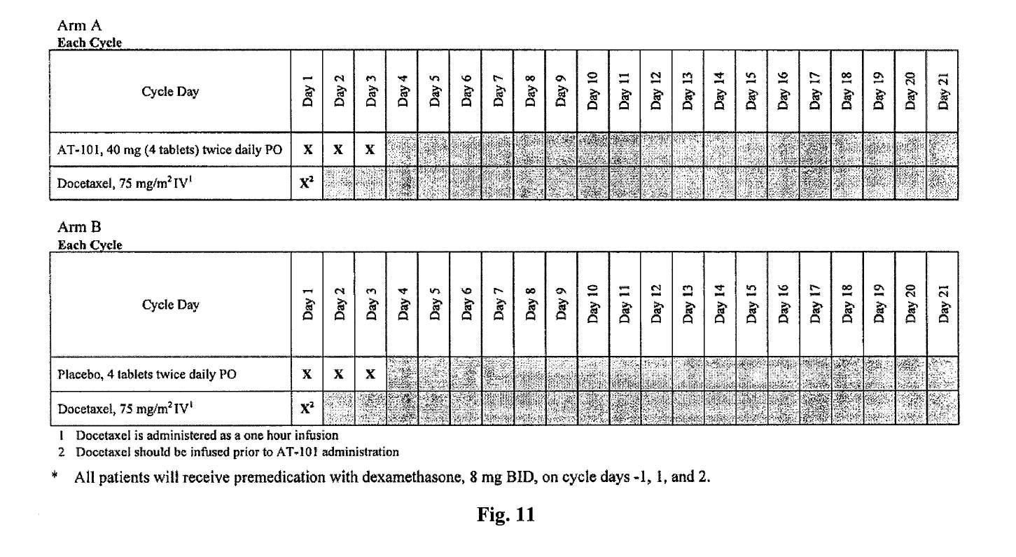

Briefly, patients were divided into two treatment arms for study: Arm A: AT-101 40 mg b.i.d. p.o. for 3 days on cycle days 1, 2, and 3, docetaxel 75 mg/m² i.v. on cycle day 1, and oral dexamethasone premedication, 8 mg b.i.d on cycle days −1, 1, and 2 of each treatment cycle. Cycle length is 21 days. AT-101 is supplied as a 10 mg tablet; and Arm B: placebo (4 tablets) b.i.d. p.o. for 3 days on cycle days 1, 2, and 3, docetaxel 75 mg/m² i.v. on cycle day 1, and oral dexamethasone premedication, 8 mg b.i.d on cycle days −1, 1, and 2 of each treatment cycle. Cycle length is 21 days. The dosing schedules are shown schematically in FIG. 11. Table 7 shows the patient demographics in the modified intent-to-treat (ITT) population. Table 8 shows the patient smoking history in the modified intent-to-treat population. Tables 9-11 show the baseline disease characteristics in the modified intent-to-treat population.

TABLE 7

| | AT-101 + | Placebo + | |
|---|---|---|---|
| | Docetaxel | Docetaxel | Overall |
| | (N = 53) | (N = 52) | (N = 105) |
| Age (years) | | | |
| n | 53 | 52 | 105 |
| Mean | 58.9 | 58.8 | 58.9 |
| Std Dev | 10.83 | 8.78 | 9.82 |
| Median | 58 | 59.5 | 59 |
| Min/Max | 26/78 | 39/74 | 26/78 |
| Age Group | | | |
| <65 | 33 (62.3%) | 37 (71.2%) | 70 (66.7%) |
| >=65 | 20 (37.7%) | 15 (28.8%) | 35 (33.3%) |
| Gender | | | |
| Female | 11 (20.8%) | 13 (25.0%) | 24 (22.9%) |
| Male | 42 (79.2%) | 39 (75.0%) | 81 (77.1%) |

[1] One patient was excluded due to no protocol treatment received.

TABLE 8

| | AT-101 + | Placebo + | |
|---|---|---|---|
| | Docetaxel | Docetaxel | Overall |
| Smoking Status | (N = 53) | (N = 52) | (N = 105) |
| Current | 31 (58.5%) | 25 (48.1%) | 56 (53.3%) |
| Never | 13 (24.5%) | 9 (17.3%) | 22 (21.0%) |
| Former | 9 (17.0%) | 18 (34.6%) | 27 (25.7%) |

[1] One patient was excluded due to no protocol treatment received.

TABLE 9

| | AT-101 + | Placebo + | |
|---|---|---|---|
| | Docetaxel | Docetaxel | Overall |
| | (N = 53) | (N = 52) | (N = 105) |
| Time Since Last | | | |
| Chemotherapy (months) | | | |
| n | 53 | 52 | 105 |
| Mean | 5.7 | 4.9 | 5.3 |
| Std Dev | 6.19 | 6.26 | 6.21 |
| Median | 2.8 | 3.2 | 3.2 |
| Min/Max | 0.9/29.0 | 0.9/40.9 | 0.9/40.9 |
| Number of Patients <3 | 27 (50.9%) | 25 (48.1%) | 52 (49.5%) |
| months | | | |
| Number of Patients >=3 | 26 (49.1%) | 27 (51.9%) | 53 (50.5%) |
| months | | | |
| Prior Surgery | | | |
| No | 37 (69.8%) | 30 (57.7%) | 67 (63.8%) |
| Yes | 16 (30.2%) | 22 (42.3%) | 38 (36.2%) |
| Prior Radiation | | | |
| No | 41 (77.4%) | 35 (67.3%) | 76 (72.4%) |
| Yes | 12 (22.6%) | 17 (32.7%) | 29 (27.6%) |
| Time Since Diagnosis | | | |
| (months) | | | |
| n | 53 | 52 | 105 |
| Mean | 13.4 | 14.8 | 14.1 |
| Std Dev | 13.34 | 18.88 | 16.26 |
| Median | 9.1 | 8.6 | 8.7 |
| Min/Max | 0.7/64.3 | 0.3/99.3 | 0.3/99.3 |
| Histological Type | | | |
| Squamous | 28 (52.8%) | 31 (59.6%) | 59 (56.2%) |
| Adenocarcinoma | 18 (34.0%) | 14 (26.9%) | 32 (30.5%) |
| Large Cell | 2 (3.8%) | 5 (9.6%) | 7 (6.7%) |
| Bronchoalveolar | 3 (5.7%) | 1 (1.9%) | 4 (3.8%) |
| Other | 2 (3.8%) | 1 (1.9%) | 3 (2.9%) |

[1] One patient was excluded due to no protocol treatment received.

TABLE 10

| | AT-101 + | Placebo + | |
|---|---|---|---|
| | Docetaxel | Docetaxel | Overall |
| | (N = 53) | (N = 52) | (N = 105) |
| Stage at Diagnosis | | | |
| I | 2 (3.8%) | 2 (3.8%) | 4 (3.8%) |
| II | 4 (7.5%) | 4 (7.7%) | 8 (7.6%) |
| IIIA | 4 (7.5%) | 7 (13.5%) | 11 (10.5%) |
| IIIB | 13 (24.5%) | 14 (26.9%) | 27 (25.7%) |
| IV | 30 (56.6%) | 25 (48.1%) | 55 (52.4%) |
| Stage at Baseline | | | |
| IIIB | 4 (7.5%) | 2 (3.8%) | 6 (5.7%) |
| IV | 49 (92.5%) | 50 (96.2%) | 99 (94.3%) |
| ECOG PS | | | |
| 0 | 2 (3.8%) | 6 (11.5%) | 8 (7.6%) |
| 1 | 44 (83.0%) | 39 (75.0%) | 83 (79.0%) |
| 2 | 7 (13.2%) | 7 (13.5%) | 14 (13.3%) |
| Number of Metastatic | | | |
| Sites (organs) | | | |
| 0 | 3 (5.7%) | 2 (3.8%) | 5 (4.8%) |
| 1 | 2 (3.8%) | 5 (9.6%) | 7 (6.7%) |
| 2 | 15 (28.3%) | 8 (15.4%) | 23 (21.9%) |
| 3 | 20 (37.7%) | 23 (44.2%) | 43 (41.0%) |
| 4 | 12 (22.6%) | 8 (15.4%) | 20 (19.0%) |
| 5 | 1 (1.9%) | 6 (11.5%) | 7 (6.7%) |
| Prior Systemic Anti- | | | |
| Cancer Regimens (all) | | | |
| 1 | 44 (83.0%) | 42 (80.8%) | 86 (81.9%) |
| 2 | 8 (15.1%) | 10 (19.2%) | 18 (17.1%) |
| 4 | 1 (1.9%) | 0 (0.0%) | 1 (1.0%) |

[1] One patient was excluded due to no protocol treatment received.

TABLE 11

| | AT-101 + | Placebo + | |
|---|---|---|---|
| | Docetaxel | Docetaxel | Overall |
| | (N = 53) | (N = 52) | (N = 105) |
| Prior Systemic Anti- | | | |
| Cancer Regimens | | | |
| (metastatic only) | | | |
| 0 | 4 (7.5%) | 3 (5.8%) | 7 (6.7%) |
| 1 | 40 (75.5%) | 39 (75.0%) | 79 (75.2%) |
| 2 | 8 (15.1%) | 10 (19.2%) | 18 (17.1%) |
| 4 | 1 (1.9%) | 0 (0.0%) | 1 (1.0%) |
| Prior Systemic Anti- | | | |
| Cancer Regimens | | | |
| (adjuvant only) | | | |
| 0 | 47 (88.7%) | 49 (94.2%) | 96 (91.4%) |
| 1 | 5 (9.4%) | 3 (5.8%) | 8 (7.6%) |
| 3 | 1 (1.9%) | 0 (0.0%) | 1 (1.0%) |
| Prior Chemotherapy | | | |
| Regimens | | | |
| 1 | 50 (94.3%) | 51 (98.1%) | 101 (96.2%) |
| 2 | 2 (3.8%) | 1 (1.9%) | 3 (2.9%) |
| 4 | 1 (1.9%) | 0 (0.0%) | 1 (1.0%) |
| Prior EGFR Inhibitor | | | |
| Therapy | | | |
| no | 44 (83.0%) | 43 (82.7%) | 87 (82.9%) |
| yes | 9 (17.0%) | 9 (17.3%) | 18 (17.1%) |

[1] One patient was excluded due to no protocol treatment received.

### Progression Free Survival

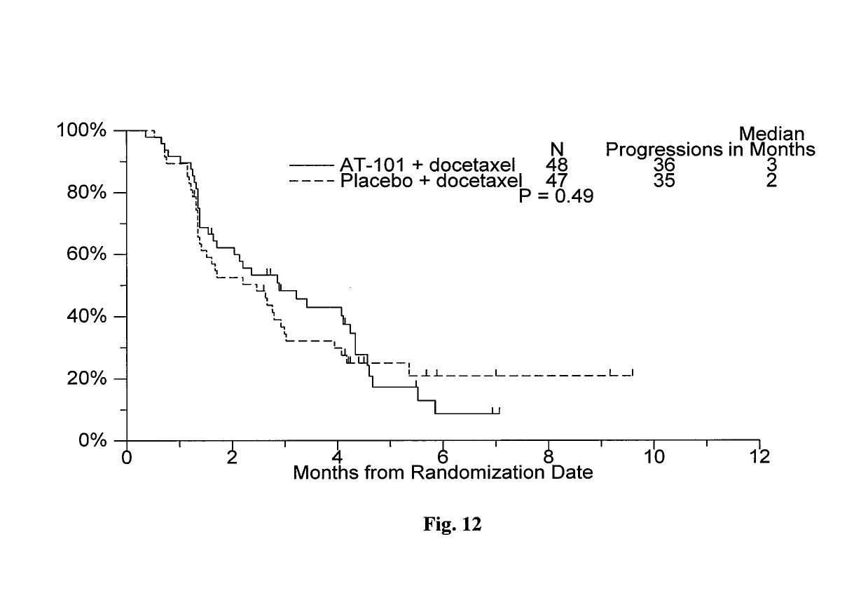

One hundred and six patients were randomized to the protocol. One patient did not receive any protocol treatment and was excluded from the analysis. In addition, five patients each randomized to AT-101 and placebo had no on-study disease assessment data or qualified PFS events. Since the effective follow-up time was null for these patients, they were also excluded from analysis. The remaining 95 patients, 48 on the AT-101 arm (Arm A) and 47 on placebo arm (Arm B), all received at least some of the assigned protocol treatment and were analyzed according to the randomized treatment. Seventy one PFS events were reported among the 95 patients. The results of this analysis are summarized in Table 12. Progression free survival Kaplan-Meier curves are presented in FIG. 12.

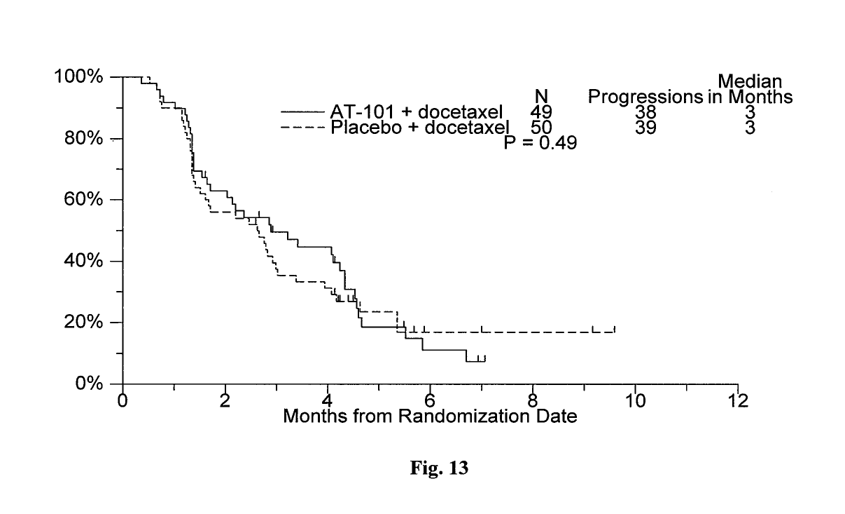

A secondary PFS analysis was also conducted for which six late deaths, two on AT-101 arm and 4 on Placebo arm, were included as events. These deaths occurred >10 weeks past the last disease assessment and, according to the Statistical Analysis Plan, could not be counted as events. PFS time was censored at the last disease assessment date for these patients in the main analysis. Four of the six patients had no on-study disease assessments and had to be excluded from the main PFS analysis. With the inclusion of these four cases, 99 patients with a total of 77 events were included in this secondary analysis. The results of this analysis are summarized in Table 13. Progression free survival Kaplan-Meier curves are presented in FIG. 13.

In summary, for the overall population of patients, there was no statistically significant improvement in PFS as assessed by investigator determined progression. However, these PFS data indicate that a subgroup of patients in the trial derived a benefit from the treatment of AT-101 in combination with docetaxel compared to docetaxel and placebo. After an initial group of patients with rapid disease progression came off of study medication at the first assessment time, a subgroup of patients on active therapy had longer PFS compared to placebo, as evidenced by a separation of the survival curves. Due to the small number of patients in this trial, very few patients are contributing to the tails of the PFS curves and these may be considered unstable.

### Overall Survival

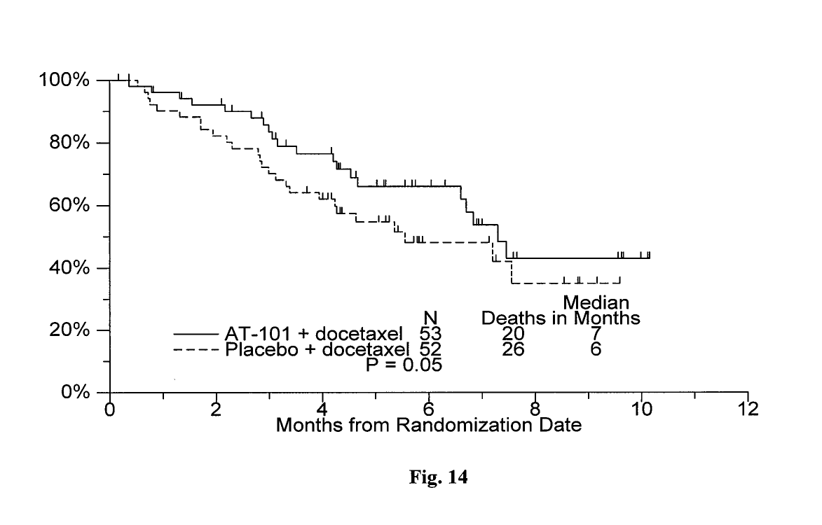

One hundred and five patients were included in the overall survival (OS) analysis, 53 patients on the AT-101 arm and 52 on the Placebo arm. One patient did not receive any protocol treatment and was excluded from the analysis for overall survival. There were a total of 46 deaths for this analysis. The results of this analysis are summarized in Table 14. Overall survival Kaplan-Meier curves are presented in FIG. 14.

Per protocol, both PFS and OS were analyzed by a logrank test stratified by the randomization stratification factors, i.e. time from last chemotherapy (≧3 months vs. <3 months) and ECOG performance status (0 or 1 vs. 2). The results are presented in the following tables and figures. On-sided p values testing for an AT-101 benefit are displayed on the figures.

These overall survival data support a clinical benefit from the treatment of NSCLC with docetaxel in combination with AT-101 compared to docetaxel alone. The survival curves separate early in the observation period and continue to separate further resulting in a hazard ratio of 0.60 with a one-sided P value of 0.05 based on the protocol specified logrank testing of the survival curves. This amounts to an estimate of a reduction of death of 40% from therapy with AT-101 compared to placebo.

TABLE 12

| | | AT 101 + | Placebo |
|---|---|---|---|
| | | Docetaxel | Docetaxel |
| | | (N = 48) [1] | (N = 47) [1] |
| Investigator | Number of Patients with Progression or Who died | 36 | 35 |
| Assessment | Number of Patients with Progression | 32 | 26 |
| | Radiographic Progression | 29 | 24 |
| | Symptomatic Progression | 3 | 2 |
| | Number of Patients with death without Progression | 4 | 9 |
| | Number of Censored Observations | 12 | 12 |
| | Quartile (Days) | | |
| | First Quartile (75th Percentile of the KM curve) | 42 | 40 |
| | Median Quartile (50th Percentile of the KM curve) | 88 | 75 |
| | Third Quartile (25th Percentile of the KM curve) | 139 | 127 |
| | Minimum, Maximum Time to Events | 11, 178 | 16, 163 |
| | Hazard Ratio (AT 101 vs. Placebo) | 1.00 | |
| | 95% CI about the Hazard Ratio | (0.62, 1.61) | |
| | 2-sided P-Value [2] | 0.99 | |
| | 1-sided P-value [2] | 0.49 | |

[1] Excluding one patient due to no protocol treatment received and 10 other patients with no events reported and no on-study disease assessments.
[2] P values are based on a stratified (by randomization stratification factors) logrank test.

TABLE 13

| | | AT 101 + | Placebo |
|---|---|---|---|
| | | Docetaxel | Docetaxel |
| | | (N = 49) [1] | (N = 50) [1] |
| Investigator | Number of Patients with Progression or Who died | 38 | 39 |
| Assessment | Number of Patients with Progression | 32 | 26 |
| | Radiographic Progression | 29 | 24 |
| | Symptomatic Progression | 3 | 2 |
| | Number of Patients with death without Progression | 6 | 13 |
| | Number of Censored Observations | 11 | 11 |
| | Quartile (Days) | | |
| | First Quartile (75th Percentile of the KM curve) | 42 | 41 |
| | Median Quartile (50th Percentile of the KM curve) | 88 | 80 |
| | Third Quartile (25th Percentile of the KM curve) | 139 | 141 |
| | Minimum, Maximum Time to Events | 11, 204 | 16, 163 |
| | Hazard Ratio (AT 101 vs. Placebo) | 0.96 | |
| | 95% CI about the Hazard Ratio | (0.60, 1.53) | |
| | 2-sided P-Value [2] | 0.84 | |
| | 1-sided P-value [2] | 0.42 | |

[1] Excluding 3 patients due to a lack of on-study data, and 8 other patients with no events reported and no on-study disease assessments. The 95 patients included in analysis all received at least some protocol treatment as randomized, i.e. was no modification to the ITT principle in this group of patients.
[2] P values are based on a stratified (by randomization stratification factors) logrank test.

TABLE 14

| | AT 101 + | Placebo |
|---|---|---|
| | Docetaxel | Docetaxel |
| | (N = 53) [1] | (N = 52) [1] |
| Number of Patients Who Died | 20 | 26 |
| Number of Censored Observations | 33 | 26 |
| Quartile (Days) | | |
| First Quartile (75th Percentile of the KM | 128 | 86 |
| curve) | | |
| Median Quartile (50th Percentile of the KM | 222 | 169 |
| curve) | | |
| Third Quartile (25th Percentile of the KM | NR | NR |
| curve) | | |
| Minimum, Maximum Time to Events | 11, 227 | 16, 230 |
| Hazard Ratio (AT 101 vs. Placebo) | 0.60 | |
| 95% CI about the Hazard Ratio | (0.33, 1.11) | |
| 2-sided P-Value [2] | 0.10 | |
| 1-sided P-value [2] | 0.05 | |

[1] Excluding patient due to protocol treatment received.
[2] P values are based on a stratified (by randomization stratification factors) logrank test

### Example 7

### Patients with NSCLC Having Squamous Cell Histology

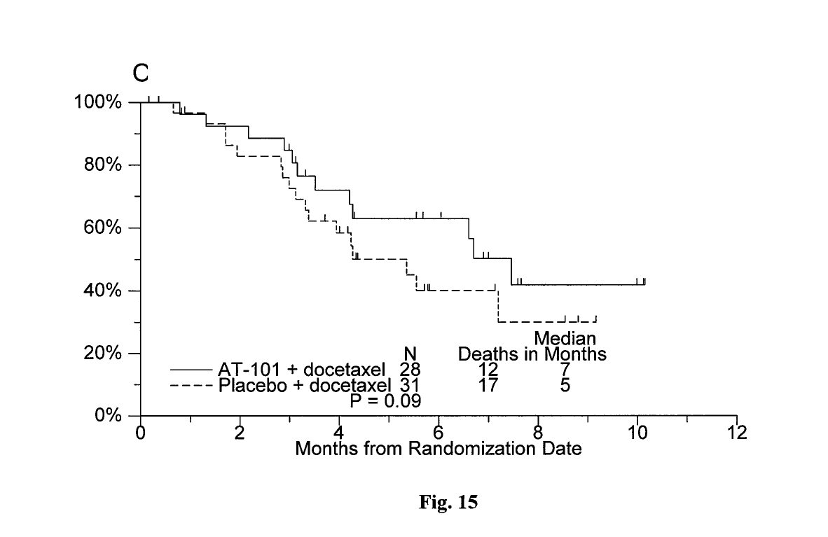

In connection with the clinical study described in Example 6, an exploratory subset analysis was performed based on performance status and tumor histology. This subset analysis demonstrated a treatment benefit in patients with an ECOG performance status of 0 or 1 and with squamous cell histology. The Kaplan-Maier survival curve for patients with squamous cell histology is presented in FIG. 15. The hazard ratio is 0.59 with a p value of 0.09. The median overall survival in the experimental and control arms was 7 and 5 months, respectively. Thus, pulsatile dose administration of AT-101 in combination with an anticancer agent may be particularly efficacious in squamous cell cancers.

### Example 8

### Reduction Adverse Events

In connection with the clinical study described in Example 6, adverse events (AEs) were compared for 52 patients who received docetaxel and placebo with 53 patients who received docetaxel and AT-101. These data are summarized in Table 15.

TABLE 15

| | |
|---|---|
| Docetaxel + Placebo | Docetaxel + AT-101 |
| N = 52 | N = 53 |

| | All, | Grade ¾, | All, | Grade ¾, |
|---|---|---|---|---|
| Adverse Event | N (%) | N (%) | N (%) | N (%) |
| Fatigue | 12 (23) | 4 (8) | 7 (13) | 2 (4) |
| Anemia | 10 (19) | 0 (0) | 9 (17) | 2 (4) |
| Neutropenia | 9 (17) | 7 (13) | 4 (8) | 3 (6) |
| Dyspnea | 9 (17) | 2 (4) | 10 (19) | 3 (6) |
| Nausea | 8 (15) | 0 (0) | 8 (15) | 1 (2) |
| Prolongation of | 7 (14) | 1 (2) | 5 (9) | 0 (0) |
| QTc | | | | |
| Anorexia | 7 (14) | 1 (2) | 4 (8) | 2 (4) |
| Asthenia | 6 (12) | 1 (2) | 7 (13) | 2 (4) |
| Non cardiac chest | 4 (8) | 1 (2) | 5 (9) | 0 (0) |
| pain | | | | |
| Peripheral sensory | 4 (8) | 0 (0) | 2 (4) | 0 (0) |
| neuropathy | | | | |
| Alopecia | 4 (8) | 0 (0) | 5 (9) | 0 (0) |
| Diarrhea | 3 (6) | 0 (0) | 4 (8) | 0 (0) |
| Vomiting | 3 (6) | 0 (0) | 4 (8) | 0 (0) |
| Troponin elevation | 3 (6) | 1 (2) | 4 (8) | 0 (0) |
| Decreased Appetite | 3 (6) | 0 (0) | 3 (6) | 0 (0) |
| Hyperglycemia | 3 (6) | 1 (2) | 4 (8) | 0 (0) |
| Arthralgia | 3 (6) | 0 (0) | 2 (4) | 0 (0) |
| Cough | 3 (6) | 0 (0) | 4 (8) | 0 (0) |
| Rash | 2 (4) | 0 (0) | 4 (8) | 0 (0) |
| Headache | 0 (0) | 0 (0) | 5 (9) | 0 (0) |

There was a decrease in the instances of any grade of fatigue, neutropenia, anorexia, and peripheral sensory neuropathy among patients who received docetaxel in combination with AT-101. Thus, administration of AT-101 to patients undergoing cancer therapy reduces the number and/or severity of adverse events the patient experiences.

### Example 9

### Clinical Study of Gossypol in Combination with Topotecan HCl for the Treatment of Small Cell Lung Cancer

An open-label, phase 1/2 study of AT-101 in combination with topotecan HCl in patients with relapsed and refractory small cell lung cancer (SCLC) was conducted. Two cohorts of patients were evaluated and included chemotherapy refractory disease (relapse within 60 days of prior chemotherapy) and chemotherapy sensitive relapsed disease (relapse 60 days or more following the completion of prior chemotherapy). The dose for the phase 2 portion of the study is AT-101 40 mg/day p.o. days 1-5 in combination with topotecan HCl 1.5 mg/day i.v. days 1-5 repeated every 21 days (one cycle). The study is currently under clinical review, however, the overall response rate (ORR) and time-to-progression (TTP) endpoints based on investigator determined responses are notably improved compared to historical controls. For the resistant cohort, the preliminary ORR (Response rate+stable disease rate) and median TTP were 60% (6/10) and 12.5 weeks, respectively. For the sensitive cohort, the preliminary ORR and median TTP were 82% (14/17) and 17.3 weeks, respectively. In a recently reported phase 3 trial of oral versus i.v. topotecan HCl in all-corners with relapsed SCLC, the ORR was 44% and median progression free survival (PFS) was 13.1 weeks (von Pawel et al., J. Clin. Oncol. 19:1743-1749 (2001)).

### Example 10

### Clinical Study of Gossypol in Combination with Docetaxel in Men with Docetaxel-Refractory CRPC

A clinical study of docetaxel and prednisone in combination with AT-101 using a pulse administration schedule in men with docetaxel-refractory castration-resistant prostate cancer (CRPC) is ongoing (see Example 4, cohort B patients). A strict definition of docetaxel refractoriness which requires that the patient have progression as measured by PSA levels, RECIST, or bone scan while receiving docetaxel (D) and prednisone (P) is being used in the study. RECIST criteria are widely-used and well known in the art. The intervention is to continue docetaxel and prednisone treatment and to add AT-101. The null hypothesis (no effect) is that there would be no responses to continuing the D/P alone in this setting. To date, 38 patients are enrolled into this cohort of the study and PSA and RECIST data have been collected on 35 patients, many who are still on-study. Three patients did not meet the strict definition of refractoriness but are included in the summary for completeness.

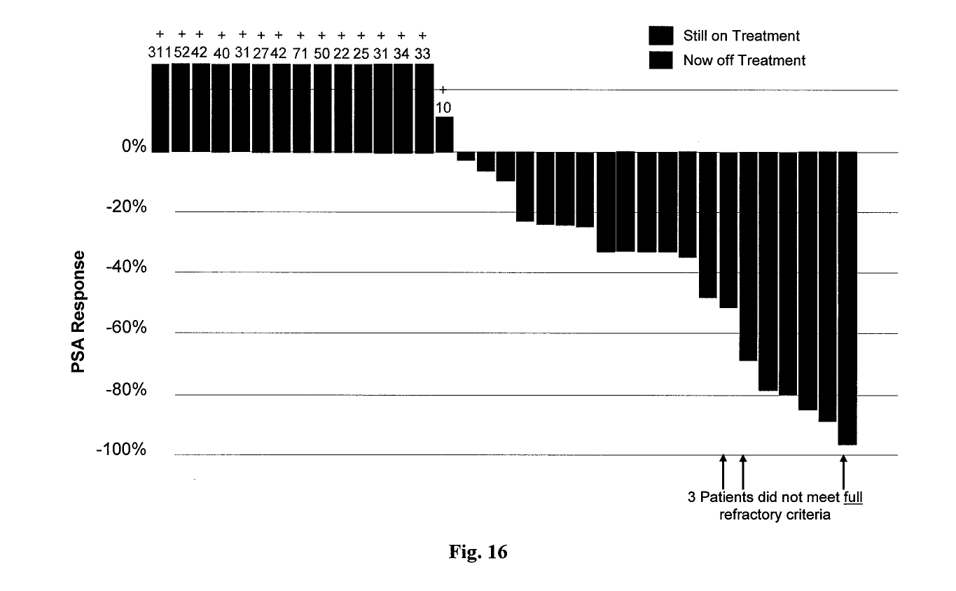

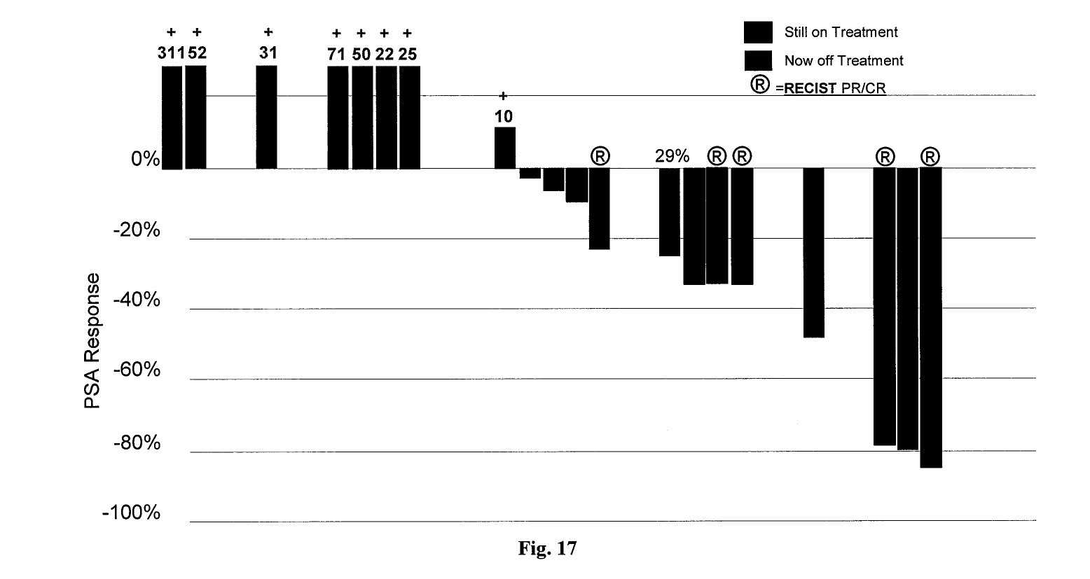

As shown in FIG. 16, 20/35 patients have had some PSA reduction, including 7/35 patients who have achieved a PSA partial response (PR) (50% or greater reduction). There are 4/32 D/P refractory patients who have had a PSA PR. There are 20 patients in this group who have measurable disease by RECIST criteria at baseline, and 6/20 have had major tumor shrinkage based on radiographic assessments, as indicated in FIG. 17. This includes one complete response (CR), 4 PRs and another patient with a 29% reduction in tumor volume. Eight out of the 35 patients have been on-study for 4 months or longer, including 3 patients who have been on-study for 6 months or longer.

These clinical data show that pulsatile dose administration of AT-101 results in higher peak and AUC concentrations which lower the threshold to apoptosis and overcomes D/P chemoresistance in some patients with CRPC. Also, pulsatile dosing of AT-101 achieves greater efficacy as compared to single agent AT-101 administration (AT-101 at 20-30 mg daily) in men with treatment naïve CRPC, where no RECIST SD (stable disease) and fewer PSA PRs were observed. Better compliance with a 3-day dosing regimen, compared to daily for 21 of 28 days, for example, is expected. Pulsatile dose administration of AT-101 was well tolerated.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

What is claimed is:

1. A method of treating or ameliorating cancer comprising administering to a patient in need thereof (−)-gossypol in combination with one or more anticancer agents, wherein:
(a) said (−)-gossypol is administered to said subject only on day 1, day 2, and day 3 of a 14-day, 21-day, or 28-day treatment cycle; or
(b) said (−)-gossypol is administered to said subject only on day 1, day 2, day 3, day 4, and day 5 of a 14-day, 21-day, or 28-day treatment cycle; and
(c) said one or more anticancer agents are administered to said subject at least on day 1 of said treatment cycle;
wherein said treatment cycle is optionally repeated one or more times.

2. The method of claim 1, wherein said cancer is selected from the group consisting of breast cancer, prostate cancer, lymphoma, skin cancer, pancreatic cancer, colon cancer, malignant melanoma, ovarian cancer, brain cancer, primary brain carcinoma, head and neck cancer, glioma, glioblastoma, liver cancer, bladder cancer, non-small cell lung cancer, head carcinoma, neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, retinoblastoma, neuroendocrine carcinoma, laryngeal cancer, and colorectal cancer.

3. The method of claim 2, wherein said cancer is selected from the group consisting of breast cancer, prostate cancer, head and neck cancer, glioblastoma, non-small cell lung cancer, esophageal carcinoma, adrenal cortex carcinoma, chronic lymphocytic leukemia, and non-Hodgkin's lymphoma.

4. The method of claim 1, wherein said cancer is a squamous cell cancer.

5. The method of claim 5, wherein said squamous cell cancer is selected from the group consisting of squamous cell skin cancer, squamous cell non-small cell lung cancer, squamous cell laryngeal cancer, squamous cell nasopharyngeal cancer, squamous cell tongue cancer, squamous cell esophagus cancer, squamous cell stomach cancer, squamous cell anal cancer, squamous cell bladder cancer, squamous cell penile cancer, squamous cell cervical cancer, squamous cell endometrium cancer, and squamous cell cancer.

6. The method of claim 1, wherein said one or more anticancer agents is selected from the group consisting of abraxane, actinomycin D, aldesleukin, alemtuzumab, alitretinoin, allopurinol, altretamine, amifostine, aminoglutethamide, anastrozole, arsenic trioxide, asparaginase, azacitidine, azathioprine, BCG live, bevacizumab, bexarotene, bicalutamide, bleomycin, bortezomib, busulfan, butazolidin, capecitabine, carboplatin, carmustine, celecoxib, cetuximab, chlorambucil, cisplatin, cladribine, clofarabine, cyclophosphamide, cytarabine, dacarbazine, dactinomycin, darbepoetin alfa, daunomycin, daunorubicin, denileukin diftitox, dexamethasone, dexrazoxane, diethylstilbestrol, docetaxel, doxorubicin, dromostanolone propionate, epirubicin, epoetin alfa, erbitux, erlotinib, estramustine, ethinyl estradiol, etoposide, exemestane, filgrastim, finasteride, floxuridine, fludarabine, 5-fluorouracil, fluoxymesterone, flutamide, fulvestrant, gefitinib, gemcitabine, gemtuzumab, goserelin acetate, hexamethylmelamine, hydroxychloroquine, hydroxyprogesterone caproate, hydroxyurea, ibritumomab, idarubicin, ifosfamide, imatinib, interferon alfa-2a, interferon alfa-2b, interleukin-2, irinotecan, ketoconazole, lapatinib, lenalidomide, letrozole, leucovorin, leuprolide, levamisole HCl, lomustine, mechlorethamine, medroxyprogesterone acetate, megestrol acetate, meloxicam, melphalan, mercaptopurine, mesna, methotrexate, methoxsalen, methylprednisolone, metronidazole, misonidazole, mithramycin, mitomycin, mitotane, mitoxantrone, nandrolone phenpropionate, nitrogen mustard, nitroimidazole, nitrosourea, nofetumomab, oblimersen sodium, oprelvekin, oxaliplatin, oxaliplatin, oxyphenbutazone, paclitaxel, pamidronate, pazopanib, pegademase, pegaspargase, pegfilgrastim, pentostatin, phenylbutazone, picoplatin, pipobroman, plicamycin, plicamycin, porfimer sodium, prednisolone, prednisone, procarbazine, procarbazine, quinacrine, raloxifene, rasburicase, rituximab, romidepsin, sargramostim, semustine, sorafenib, streptozocin, sunitinib, talc, tamoxifen, temozolomide, teniposide, testolactone, testosterone propionate, thalidomide, thioguanine, thiotepa, tiripazamine, topotecan HCl, toremifene, tositumomab, trastuzumab, tretinoin, trimethoprim/sulfamethoxazole, uracil mustard, valrubicin, vinblastine, vincristine, vindesine, vinorelbine, zoledronic acid, and radiation therapy.

7. The method of claim 6, wherein said one or more anticancer agents is selected from the group consisting of docetaxel, paclitaxel, topotecan HCl, erlotinib, lenalidomide, cisplatin, etoposide, cetuximab, rituximab, carboplatin, oxaliplatin, 5-fluorouracil, and radiation.

8. The method of claim 1, wherein said patient is chemoresistant to said one or more anticancer agents.

9. The method of claim 1, wherein:
(a) said (−)-gossypol is administered to said patient only on day 1, day 2, and day 3;
(b) said cancer is prostate cancer;
(c) said treatment cycle is 21 days; and
(d) said one or more anticancer agents are selected from the group consisting of docetaxel; docetaxel and prednisone; mitoxantrone; and mitoxantrone and prednisone.

10. The method of claim 9, wherein said one or more anticancer agents is docetaxel and prednisone.

11. The method of claim 1, wherein:
(a) said (−)-gossypol is administered to said patient only on day 1, day 2, and day 3;
(b) said cancer is non-small cell lung cancer;
(c) said treatment cycle is 21 days; and
(d) said one or more anticancer agents are selected from the group consisting of docetaxel; cisplatin and etoposide; and erlotinib.

12. The method of claim 1 wherein:
(a) said (−)-gossypol is administered to said patient only on day 1, day 2, and day 3;
(b) said cancer is selected from the group consisting of breast cancer, non-small cell lung cancer, ovarian carcinoma, and Kaposi's sarcoma;
(c) said treatment cycle is 21 days or 28 days; and
(d) said one or more anticancer agents is paclitaxel.

13. The method of claim 1, wherein:
(a) said (−)-gossypol is administered to said patient only on day 1, day 2, and day 3;
(b) said cancer is selected from the group consisting of multiple myeloma and chronic lymphocytic leukemia;
(c) said treatment cycle is 21 days; and
(d) said one or more anticancer agents is lenalidomide.

14. The method of claim 1, wherein:
(a) said (−)-gossypol is administered to said patient only on day 1, day 2, and day 3;
(b) said cancer is head and neck cancer;
(c) said treatment cycle is 21 days; and
(d) said one or more anticancer agents are selected from the group consisting of cisplatin; cisplatin and radiation; erbitux; and carboplatin, 5-fluorouracil, and cetuximab.

15. The method of claim 1, wherein:
(a) said (−)-gossypol is administered to said patient only on day 1, day 2, and day 3;
(b) said cancer is follicular lymphoma;
(c) said treatment cycle is 21 days; and
(d) said one or more anticancer agents are rituximab, cyclophosphamide, doxorubicin, and vincristine.

16. The method of claim 1, wherein:
(a) said (−)-gossypol is administered to said patient only on day 1, day 2, day 3, day 4, and day 5;
(b) said cancer is non-small cell lung cancer;
(c) said treatment cycle is 21 days; and
(d) said one or more anticancer agents is topotecan HCl.

17. A method of reducing the number of one or more adverse events, the severity of one or more adverse events, or combination thereof, in a patient undergoing cancer therapy comprising administering (−)-gossypol to said patient at least on day 1, day 2, and day 3 of a treatment cycle.

18. The method of claim 17, wherein said adverse events are selected from the group consisting of fatigue, neutropenia, anorexia, and peripheral sensory neuropathy.

19. A method of treating or ameliorating prostate cancer or non-small cell lung cancer comprising administering to a patient in need thereof (−)-gossypol in combination with docetaxel, wherein:
(a) about 40 mg of said (−)-gossypol is orally administered to said patient twice-a-day only on day 1, day 2, and day 3 of a 21-day treatment cycle; and
(b) docetaxel is intravenously administered to said patient on day 1 of said treatment cycle.

20. The method of claim 19, wherein said non-small cell lung cancer is squamous cell non-small cell lung cancer.
